(19) United States
(12) Patent Application Publication
(10) Pub. No.: US 2003/0226154 A1
(43) Pub. Date: Dec. 4, 2003

(54) Pathophysiology associated with a single gene (MASS 1) mutation underlying the robust audiogenic seizure phenotype in frings mice

(76) Inventors: Brian Klein, Salt Lake City, UT (US); H. Steve White, Salt Lake City, UT (US); Louis Ptacek, Salt Lake City, UT (US)

Correspondence Address: MADSON & METCALF, GATEWAY TOWER WEST, SUITE 900, 15 WEST SOUTH TEMPLE, SALT LAKE CITY, UT, 84101

(21) Appl. No.: 10/423,483

(22) Filed: Apr. 25, 2003

Related U.S. Application Data
(63) Continuation-in-part of application No. 10/220,587, filed on Sep. 3, 2002.
Application No. 10/220,587 is a 371 of international application No. PCT/US01/06962, filed on Mar. 2, 2001.
(60) Provisional application No. 60/187,209, filed on Mar. 3, 2000.
(60) Provisional application No. 60/222,898, filed on Aug. 3, 2000.

(51) Int. Cl. A01K 67/027; C12Q 1/68; C07H 21/04; C12N 5/06

(52) U.S. Cl. 800/3; 800/18; 435/6; 536/23.2; 435/354

(57) ABSTRACT

The present invention relates to a novel gene which is associated with audiogenic seizures in mice. The gene is known as the Monogenic Audiogenic Seizure-susceptible gene or mass1. The product of the mass1 gene is designated MASS1. Nucleic acid molecules that encode for MASS I have been identified and purified. The sequence of murine mass1 can be found at SEQ ID NO: 1, and the sequence of human mass1 can be found at SEQ ID NO: 3. Mammalian genes encoding a MASS1 protein are also provided. The invention also provides recombinant vectors comprising nucleic acid molecules that code for a MASS1 protein. These vectors can be plasmids. In certain embodiments, the vectors are prokaryotic or eukaryotic expression vectors. The nucleic acid coding for MASS1 can be linked to a heterologous promoter. The invention also relates to transgenic animals in which one or both alleles of the endogenous mass1 gene is mutated. The invention further relates to a hearing impairment associated with the Frings MASS1 mutation. More specifically, the invention characterizes a moderate and non-progressive hearing impairment which leads to the development of audiogenic seizures.

### CROSS-REFERENCE TO RELATED APPLICATIONS

This application is a continuation in part application related to U.S. patent application Ser. No. 10/220,587, which is a United States nationalization of International Patent Application No. PCT/US01/06962, entitled “MASS1 Gene, A Target for Anticonvulsant Drug Development,” which is related to and claims the benefit of U.S. Provisional Application Serial No. 60/222,898 of Louis J. Ptacek, H. Steve White, Ying-Hui Fu, and Shana Skradski filed Aug. 3, 2000 and entitled “Human mass1 Gene,” and U.S. Provisional Application Serial No. 60/187,209 of Louis J. Ptacek, H. Steve White and Ying-Hui Fu, filed Mar. 3, 2000 and entitled “Novel Epilepsy Gene Is a Target for Anticonvulsant Drug Development,” which are each incorporated herein by this reference.

### STATEMENT REGARDING FEDERALLY SPONSORED RESEARCH OR DEVELOPMENT

This invention was made with Government support under Grant Numbers N01INS42311 and R01NS38616 awarded by the National Institutes of Health. The Government has certain rights to this application.

### BACKGROUND OF THE INVENTION

The present invention relates to the isolation and characterization of a novel gene relating to epilepsy. More specifically, the invention relates to the isolation and characterization of the Monogenic Audiogenic Seizure-susceptible gene, hereinafter mass1 gene, and the characterization of a hearing impairment associated with the MASS1 mutation found in Frings mice.

Epilepsy is a common neurological disorder that affects nearly 2.5 million people in the United States. Epilepsy is characterized by recurrent seizures resulting from a sudden burst of electrical energy in the brain. The electrical discharge of brain cells causes a change in a person's consciousness, movement, and/or sensations. The intensity and frequency of the epileptic seizures varies from person to person.

Epilepsies in humans can be separated into two forms, symptomatic and non-symptomatic. Symptomatic epilepsy is a seizure disorder related to a known cause such as metabolic disease, brain malformations, or brain tumors. In these cases, seizures presumably occur because of a very abnormal focus (or foci) in the brain. Genetic models of symptomatic epilepsy include the weaver mouse (wv), in which a mutation of the G protein-gated inwardly rectifying potassium channel GIRK2 results in neuro-developmental abnormalities and seizures. Signorini, S. et al. (1997), Proc Natl Acad Sci USA 94: 923-7. Fragile X-associated protein knock-out mice have a neurodevelopmental syndrome with lowered thresholds to audiogenic seizures. Musumeci, S. A. et al.(2000), Epilepsia 41: 19-23. Audiogenic seizures can also be induced in seizure-resistant mice such as C57BL/6 by priming with an earlier noise exposure, suggesting that seizure-susceptibility can be influenced by multiple genetic and environmental factors. Henry, K. R. (1967), Science 158: 938-40.

Non-symptomatic epilepsies are defined when no structural or metabolic lesions are recognized and the patients have no other neurological findings between seizures. This latter group of patients is more likely to have primary neuronal hyperexcitability that is not caused by metabolic, developmental or structural lesions. Molecular characterization of electrical hyperexcitability in human muscle diseases led to the hypothesis that such disorders might be the result of mutations in neuronal ion channels, the primary determinants of neuronal membrane excitability. Ptacek, L. J. et al. (1991), Cell 67: 1021-7.

All non-symptomatic human epilepsy syndromes and genetic mouse seizure models that have been characterized at a molecular level are caused by mutations in ion channels. Ptacek, L. J. (1999), Semin Neurol 19: 363-9; Jen, J. & L. J. Ptacek (2000), Channelopathies: Episodic Disorders of the Nervous System. Metabolic and Molecular Bases of Inherited Disease. C. R. Schriver, A. L. Beaudet, W. S. Sly and D. Valle. New York, McGraw-Hill. pp. 5223-5238; Noebels, J. L. (2000), The Inherited Epilepsies. Metabolic and Molecular Bases of Inherited Disease. C. R. Schriver, A. L. Beaudet, W. S. Sly and D. Valle. New York, McGraw-Hill. pp 5807-5832. Some patients with febrile seizures have been recognized to have mutations in sodium channel α and β1 subunits while some patients with epilepsy and episodic ataxia were shown to have calcium channel β-subunit mutations. Wallace, R. H. et al. (1998), Nat Genet 19: 366-70; Escayg, A. et al. (2000), Am J Hum Genet 66: 1531-9; Escayg, A. et al. (2000), Nat Genet 24: 343-5. The voltage-gated potassium channel genes KCNQ2 and KCNQ3, when mutated, result in benign familial neonatal convulsions. Biervert, C. et al. (1998), Science 279: 403-6; Charlier, C. et al. (1998), Nat Genet 18: 53-5; Singh, N. A. et al. (1998), Nat Genet 18: 25-9. Ligand-gated channels can also result in epilepsy as demonstrated by mutations in the α4 subunit of the neuronal nicotinic acetylcholine receptor that result in autosomal dominant nocturnal frontal lobe epilepsy. Steinlein, O. K. et al. (1995), Nat Genet 11: 201-3. In mice, the α, β and γ subunits of the voltage-sensitive calcium channel have been associated with the tottering (tg), lethargic (lh) and stargazer (stg) models of absence seizures. Fletcher, C. F. et al. (1996), Cell 87: 607-17; Burgess, D. L. et al. (1997), Cell 88: 385-92; Letts, V. A. et al. (1998), Nat Genet 19: 340-7. Finally, audiogenic seizure-susceptibility has been characterized in a mouse knockout model of the 5-HT_{2C} receptor; homozygous mice have audiogenic seizures and altered feeding behavior. Tecott, L. H. et al. (1995), Nature 374: 542-6; Brennan, T. J. et al. (1997), Nat Genet 16: 387-90.

The Frings mouse represents one of many strains of mice and rats that are sensitive to audiogenic seizures (AGS). These AGS-susceptible rodents represent models of generalized reflex epilepsy and include the well-studied DBA/2 mouse and GEPR-9 rat. The Frings mouse seizure phenotype is similar to other described audiogenic seizures and is characterized by wild running, loss of righting reflex, tonic flexion and tonic extension in response to high intensity sound stimulation. Schreiber, R. A. et al. (1980), Genet 10: 537-43. This strain was characterized 50 years ago when it arose as a spontaneous mutation on the Swiss Albino background. Frings, H. et al. (1951), J Mammal 32: 60-76. Selective inbreeding for seizure-susceptibility produced the current homozygous Frings strain with >99% penetrance of audiogenic seizures. The Frings mouse seizure phenotype was due to the autosomal recessive transmission of a single gene.

Audiogenic seizures have been observed in polygenic rodent models, such as the DBA/2 mouse and GEPR-9 rat. Collins, R. L. (1970), Behav Genet 1: 99-109; Seyfried, T. N. et al. (1980), Genetics 94: 701-718; Seyfried, T. N. & G. H. Glaser (1981), Genetics 99: 117-126; Neumann, P. E. & T. N. Seyfried (1990), Behav Genet 20: 307-23; Neumann, P. E. & R. L. Collins (1991), Proc Natl Acad Sci USA 88: 5408-12; Ribak, C. E. et al. (1988), Epilepsy Res 2: 345-55. While no genes associated with audiogenic seizures in spontaneous mutant models have been cloned, three putative loci associated with seizure-susceptibility in the DBA/2 mouse (asp1, asp2, and asp3) have been mapped to chromosomes 12, 4, and 7, respectively. Neumann & Seyfried, supra; Neumann, P. E. & R. L. Collins, supra. As a monogenic audiogenic seizures model, the Frings mice provided a unique opportunity for cloning and characterization of an audiogenic seizures gene. The Frings mice are an important naturally occurring monogenic model of a discrete non-symptomatic epilepsy and provide significant information on a novel mechanism of seizure-susceptibility as well as central nervous system excitability in general.

In light of the foregoing, it will be appreciated that it would be an advancement in the art to identify and characterize nucleic acid sequences that are associated with the monogenic AGS susceptibility in Frings mice. It would be a further advancement to identify and characterize the human orthologue of this gene. It would be a further advancement if the nucleic acid sequences could provide additional understanding of how epileptic seizures are triggered in disease. It would be a further advancement to provide a transgenic animal model wherein the endogenous gene associated with the Frings phenotype is mutated. In addition, it would be an advancement in the art to characterize a hereditary hearing impairment associated with the Frings mass1 mutation.

Such nucleic acid sequences, animals, and hearing impairment phenotypes are disclosed and claimed herein.

### BRIEF SUMMARY OF THE INVENTION

The present invention relates to an isolated novel gene which has been imputed in audiogenic seizure-susceptibility in mice known as the mass1 gene. Provided herein are nucleic acid molecules that encode the MASS1 protein. The nucleic acid molecules of the present invention may also comprise the nucleotide sequence for human mass1 (SEQ ID NO: 3) and murine mass1 (SEQ ID NO: 1). In certain other embodiments, the present invention provides nucleic acid molecules that code for the amino acid sequence of human MASS1 (SEQ ID NO: 4) and murine MASS1 (SEQ ID NO: 2). The invention also provides nucleic acid molecules complementary to the nucleic acid molecules of SEQ ID NO: 3 and SEQ ID NO: 1. The invention also relates to other mammalian mass1 genes and MASS1 proteins.

The present invention also relates to an isolated nucleic acid having at least 15 consecutive nucleotides as represented by a nucleotide sequence selected from the nucleotides of the murine mass1 gene (SEQ ID NO: 1) and the nucleotides of the human mass1 gene (SEQ ID NO: 3). A nucleotide having in the range from about 15 to about 30 consecutive nucleotides as represented by a nucleotide sequence selected from the nucleotides of the murine mass1 gene (SEQ ID NO: 1) and the nucleotides of the human mass1 gene (SEQ ID NO: 3) is also within the scope of the present invention.

The present invention also provides recombinant vectors comprising nucleic acid molecules that code for MASS1. These recombinant vectors may be plasmids. In other embodiments, these recombinant vectors are prokaryotic or eukaryotic expression vectors. The nucleic acid coding for MASS1 may also be operably linked to a heterologous promoter. The present invention further provides host cells comprising a nucleic acid that codes for MASS1.

The present invention also relates to a transgenic mammal with a mutation in one or both alleles of the endogenous mass1 gene. The mutation in one or both of the endogenous mass1 genes may result in a mammal with a seizure-susceptible phenotype. The transgenic mammal of the present invention may be a mouse. The mutation may result from the insertion of a selectable marker gene sequence or other heterologous sequence into the mammal's genome by homologous recombination. The invention also provides cells derived from the transgenic mammal.

The present invention further characterizes a hearing impairment phenotype associated with the Frings mass1 mutation. Specifically, Auditory Brainstem Response (ABR) testing was used to characterize a moderate and non-progressive hearing impairment in Frings mice. The development of audiogenic seizures as a result of the mass1-specific hearing impairment is also described.

These and other advantages of the present invention will become apparent upon reading the following detailed description and appended claims.

### BRIEF DESCRIPTION OF THE SEVERAL VIEWS OF THE DRAWING

A more particular description of the invention briefly described above will be rendered by reference to the appended drawings and graphs. These drawings and graphs only provide information concerning typical embodiments of the invention and are not therefore to be considered limiting of its scope.

FIG. 1 shows a linkage map of the mass1 locus initially defined by markers D13Mit126 and D13Mit200. Markers D13Mit69, 97, and 312 (enclosed in rectangles) were used to genotype the F2 progeny. The estimated genetic distances are shown. The location of candidate genes Nhe3, Dat1, and Adcy2 are indicated. The map inset represents the large-scale physical map of the mass1 interval spanned by yeast artificial chromsomes (YACs). SLC10 and SLC11 are novel SSLP markers, and the others are STS markers.

FIG. 2 is a fine-scale physical map of the mass1 interval defined by bacterial artificial chromosomes (BACs) and cosmids. SLC- numbers between 10 and 100 are novel SSLP markers, and SLC- numbers 100 to 200 are novel STS markers. The bars above the map represent the genotypes of the nearest recombinant mice. The gray bars represent regions where the mice are recombinant, black filled bars are regions where the mice are nonrecombinant, and white filled bars are regions where the markers were not informative. The final mass1 interval was spanned by cosmids C13A and C1B, and the complete genomic sequence was generated between the markers SLC20 and SLC14. The alignment of the mass1 exons that were identified from the sequence are shown at the bottom.

FIG. 3 is a diagram of the mass1 genomic structure showing three putative transcripts and exons that are included in each transcript. The short transcript, mass1.3, has putative 5′ untranslated sequence leading into exon 22. Exon 7a and 7b represent two alternate exons that have been identified in mouse brain cDNA. The medium transcript, mass1.2, has putative 5′ untranslated sequence leading into exon 7b, and the longest transcript, mass1.1, has only been shown to contain exon 7a. A long and short splice variant was identified in exon 27 (27L and 27S). The 27S variant removes 83 base pairs and changes the reading frame.

FIG. 4A illustrates expression analysis of the mass1 gene by RT-PCR in different tissue and cell RNA samples using primers from exons 23 and 24. Analysis of mass1 in multiple tissue RNA samples of a CF1 mouse shows expression is primarily in the brain, kidney, and lung, and not in the other tissues listed.

FIG. 4B illustrates further expression analysis of the mass1 gene by RT-PCR using brain RNA. Mass1 expression was detected in all regions of the brain tested.

FIG. 4C illustrates expression analysis by RT-PCR of the mass1 gene with pooled cultured cortical neuron RNA and cultured astrocyte RNA compared to whole brain. The mass1 specific primers span intron 23 and the expected product size was 487 base pairs. The β-actin primers also spanned two exons and the expected product size is 327 base pairs. The ladder is in 100 base pair increments.

FIG. 5A is a sequence chromatogram of the exon 27 segment from C57BL/6J and Frings DNA. The sequence chromatogram illustrates the identification of a single base pair deletion found in exon 27 of mass1 sequence of Frings mice. The Frings mouse DNA contains a single G deletion at nucleotide 7009.

FIG. 5B illustrates high resolution gel electrophoresis of PCR products from a 150 base pair segment of exon 27 encompassing 7009ΔG, showing that none of the seizure-resistant and seizure-susceptible control mouse DNA samples harbor the deletion present in the Frings mouse.

FIG. 6 illustrates the conceptual amino acid translation of the mass1.1 transcript (SEQ ID NO: 5). The 18 MASS1 repetitive motifs are boxed with a solid line and the 2 less conserved possible repeats are boxed with a dashed line. The putative multicopper oxidase I domain is underlined. The valine → stop mutation in the Frings MASS1 protein is located at amino acid number 1072 marked with the “*”.

FIG. 7 illustrates the amino acid sequence alignment of the MASS1 repeats. (SEQ ID NOS: 6-23). The first 18 lines represent the well conserved amino acid repeat motif found in MASS1. Positions of highly conserved amino acids are shaded gray. The next line shows the consensus sequence for the MASS1 repeat (SEQ ID NO: 24), and below it are the sequences of the Na⁺/Ca²⁺ exchanger (β1 and β2) segments that share homology with the MASS1 repeat (SEQ ID NOS: 25 & 26). Also shown is a homologous region of the very large G-protein coupled receptor-1 (Accession 55586) (SEQ ID NO: 27). The boxed segment outline the DDD motif that has been shown to be a Ca²⁺ binding site in the Na⁺/Ca²⁺ exchanger β1 segment.

FIG. 8 illustrates auditory brainstem response thresholds for genetically audiogenic seizure-susceptible and resistant mouse strains. Auditory brainstem response thresholds were measured for click stimulus, 10 kHz, 16 kHz and 22 kHz tone stimuli at various age-points. Results from male and female mice of each strain were combined. The mice with the Frings mass1 alleles (Frings, BUB/bnJ and congenic) display elevated mean hearing thresholds with each acoustic stimulus, even at the earliest age-points tested. The Frings mice display little progression of their auditory brainstem response thresholds until PND 580. In contrast, the BUB/bnJ, congenic and DBA/2J mice display progression of auditory brainstem response thresholds by PND 30. S.D.=standard deviation.

FIG. 9 shows mean auditory brainstem response (ABR) thresholds for the click stimulus in the different mouse strains at various age-points. The error bars represent standard deviation. The Frings, BUB/bnJ and congenic mice, which possess the mass1 deletion, displayed elevated ABR thresholds at the earliest age-points tested. The BUB/bnJ, congenic and DBA/2J mice displayed increasing ABR with age. The C57BL/6J and SWR/Bm displayed normal ABR thresholds.

FIG. 10 illustrates mean auditory brainstem response (ABR) thresholds for the 10 kHz tone stimulus in the different mouse strains at various age-points. The error bars represent standard deviation. The Frings, BUB/bnJ and congenic mice which possess the mass1 deletion displayed elevated ABR thresholds at the earliest age-points tested. The elevated ABR thresholds continued to increase for the BUB/bnJ and congenic mice.

FIG. 11 illustrates mean auditory brainstem response (ABR) thresholds for the 16 kHz tone stimulus in the different mouse strains at various age-points. The error bars represent standard deviation. The Frings, BUB/bnJ and congenic mice, which possess the mass1 deletion, displayed elevated ABR thresholds at the earliest age-points tested. The BUB/bnJ, congenic and DBA/2J mice displayed increasing ABR with age. The C57BL/6J and SWR/Bm displayed normal ABR thresholds.

FIG. 12 illustrates mean auditory brainstem response (ABR) thresholds for the 22 kHz tone stimulus in the different mouse strains at various age-points. The error bars represent standard deviation. The Frings, BUB/bnJ and congenic mice, which possess the mass1 deletion, displayed elevated ABR thresholds at the earliest age-points tested. The BUB/bnJ, congenic, C57BL/6J and DBA/2J mice displayed increasing ABR with age. The SWR/Bm displayed normal ABR thresholds.

FIG. 13 illustrates audiogenic seizure (AGS) severity of various mouse strains in response to an 11 kHz tone or electric bell stimulus. Mice were exposed to either an 11 kHz tone stimulus or an electric bell for 60 s at 110 dB. The number of mice from each group is shown that displayed the following AGS scores: 0=no response, 1=wild running less than 10 s, 2=wild running greater than 10 s or two bouts of wild running, 3=wild running and loss of righting reflex, 4=clonic seizure, 5=tonic extension. Results from male and female mice of each strain were combined. The Frings mice displayed the highest level of maximal tonic seizures that persists even to PND 328. The BUB/bnJ, DBA/2J and congenic mice display AGS-susceptibility that sharply declines within two months postnatal. The C57BL/6J and SWR/Bm mice are audiogenic seizure- resistant.

FIG. 14 illustrates the percent of tested mice displaying maximal tonic audiogenic seizure (AGS) at various age-points. The Frings, BUB/bnJ, DBA/2J and congenic mice represent genetically AGS susceptible strains and the C57BL/6J and SWR/Bm are resistant strain. Maximal AGS sensitivity sharply declines within 60 days postnatal for each of the genetically susceptible strains except for the Frings mice which develop and maintain a very high level of maximal AGS sensitivity.

FIG. 15 illustrates audiogenic seizure (AGS) severity in kanamycin-treated C57BL/6J mice in response to an electric bell stimulus. Kanamycin-treated C57BL/6J mice received a daily dose of 400 mg/kg i.p. kanamycin between PND 5 and PND 21. For AGS testing, mice were exposed to an electric bell for 60 s at 110 dB. The number of mice at each age-point is shown that displayed the following AGS scores: 0=no response, 1=wild running less than 10 s, 2=wild running greater than 10 s or two bouts of wild running, 3=wild running and loss of righting reflex, 4=clonic seizure, 5=tonic extension. Results from male and female mice were combined. The kanamycin-treated mice (2/5) displayed tonic audiogenic seizures at PND29. The severity of AGS displayed by the kanamycin-treated mice declined with age.

FIG. 16 illustrates audiogenic seizure (AGS) severity of noise-primed C57BL/6J mice in response to an electric bell stimulus. C57BL/6J mice were noise primed using an electric bell at 110 dB for 30 s or 120 s. For AGS testing, mice were exposed to an electric bell for 60 s at 110 dB. The number of mice at each age-point is shown that displayed the following AGS scores: 0=no response, 1=wild running less than 10 s, 2=wild running greater than 10 s or two bouts of wild running, 3=wild running and loss of righting reflex, 4=clonic seizure, 5=tonic extension. Results from male and female mice were combined. The C57BL/6J mice noise primed on PND 18 displayed the highest level of maximal tonic AGS activity. Mice noise primed at PND 19 and PND 20 displayed intermediate AGS scores, but not tonic extension. The PND 16 noise primed mice did not display any AGS activity at the age-points tested.

FIG. 17 illustrates auditory brainstem response (ABR) thresholds of experimentally induced audiogenic seizure-susceptible C57BL/6J mice. ABR thresholds were measured for click stimulus, 10 kHz, 16 kHz and 22 kHz tone stimuli at the indicated age-points. Results from male and female mice of each strain were combined. Kanamycin-treated mice received a daily dose of 400 mg/kg i.p. between PND 5 and PND 21. The kanamycin-treated mice display elevated ABR thresholds at all the acoustic stimuli tested at PND 19 and 24. Noise-primed mice were exposed to an electric bell (110 dB) for 30 or 120 s. The noise-primed mice displayed elevated ABR thresholds at the 16 kHz and 22 kHz stimuli. However, the increased ABR thresholds declined at each age-point tested after noise priming, demonstrating a transient hearing loss. S.D.=standard deviation.

FIG. 18 shows the templates utilized for quantifying c-Fos immunoreactive cells and analyzing the ratio for pixel area for the A) 11 kHz stimulus, B) 16 kHz stimulus, and C) 22 kHz stimulus (adapted from Franklin and Paxinos, 1997), D) The tonotopic response domains determined electrophysiologically in the inferior colliculus (adapted from Stiebler and Ehret, 1985). CIC=central nucleus inferior colliculus, DCIC=dorsal nucleus inferior colliculus, ECIC=external nucleus inferior colliculus.

FIG. 19 illustrates the pattern of c-Fos immunoreactive response following a prolonged, sub-audiogenic seizure (AGS) threshold tone stimulation (11 kHz, 80 dB). Images are from genetically AGS-susceptible: A) Frings mice and B) DBA/J mice and AGS-resistant; C) SWR/J mice and D) CF1 mice. The AGS-susceptible mice displayed more intense c-Fos immunoreactivity focused in the 11 kHz tonotopic domain.

FIG. 20 illustrates the pattern of c-Fos immunoreactive response following a prolonged, sub-audiogenic seizure (AGS) threshold tone stimulation (11 kHz, 80 dB) in: A) genetically AGS-susceptible congenic mice; B) AGS-resistant C57BL/6J mice; C) C57BL/6J noise-primed mice that developed AGS-susceptibility; and D) C57BL/6J noise-primed mice that did not develop AGS-susceptibility. The AGS-susceptible mice displayed c-Fos immunoreactivity more focused in the 11 kHz tonotopic domain.

FIG. 21 illustrates the mean number of c-Fos positive cells in the tonotopic domain following an 11 kHz sub-seizure threshold (80 dB) tone stimulation. Error bars represent standard error of the mean. Statistical significance was determined using one-way ANOVA and Tukey's post-hoc analysis at p<0.05. (*) The Frings mice displayed significantly greater c-Fos positive cell counts than all the other groups. (†) The DBA/J mice were significantly greater than the SWR/Bm, C57BL/6J and congenic mice. (‡) The audiogenic seizure-susceptible noise-primed C57BL/6J mice displayed significantly higher c-Fos positive cell counts compared to the CF1, SWR/Bm, C57BL/6J and C57BL/6J noise-primed but audiogenic seizure negative mice. FRI=Frings mice, DBA=DBA/2J mice, CF1=CF1 mice, SWR=SWR/Bm mice, C57=C57BL/6J mice, CON=Congenic mice.

FIG. 22 illustrates the ratio of pixel area of c-Fos staining for the tonotopic domain compared to the average of the areas immediately above and below following an 11 kHz sub-seizure threshold (80 dB) tone stimulation. Error bars represent standard error of the mean. Statistical significance was determined using one-way the Kruskal-Wallis test and Dunn's post-hoc analysis at P<0.05. (*) The Frings mice displayed a significantly higher ratio of staining in the tonotopic domain compared to the CF1, C57BL/6J and SWR/Bm mice. (†) DBA/2J mice displayed a significantly higher ratio compared to the CF1 and SWR/Bm mice. (‡) The audiogenic seizure-susceptible noise-primed C57BL/6J mice displayed a significantly higher ratio of straining in the tonotopic domain compared to the CF1, SWR/Bm and C57BL/6J mice. FRI=Frings mice, DBA=DBA/2J mice, CF1=CF1 mice,SWR=SWR/Bm mice, C57=C57BL/6J mice, CON=Congenic mice.

FIG. 23 illustrates the pattern of c-Fos immunoreactive response following a prolonged, sub-audiogenic seizure (AGS) threshold tone stimulation (16 kHz, 78 dB) in: A) genetically AGS-susceptible Frings mice, and AGS-resistant; B) CF1 mice and C) C57BL/6J mice. The AGS-susceptible Frings mice displayed more intense c-Fos immunoreactivity focused in the 16 kHz tonotopic domain.

FIG. 24 illustrates the pattern of c-Fos immunoreactive response following a prolonged, sub-audiogenic seizure (AGS) threshold tone stimulation (22 kHz, 80 dB) in: A) genetically AGS-susceptible Frings mice, and AGS-resistant; B) CF1 mice and C) C57BL/6J mice. The AGS-susceptible Frings mice displayed more intense c-Fos immunoreactivity focused in the 22 kHz tonotopic domain.

FIG. 25 illustrates the mean number of c-Fos positive cells in the tonotopic domain following a 16 kHz and 22 kHz sub-seizure threshold (80 dB) tone stimulation. Error bars represent standard error of the mean. Statistical significance was determined using one-way ANOVA with Tukey's post-hoc analysis at p<0.05. (*) The Frings mice displayed significantly greater cell counts than the CF1 and C57BL/6J mice at 16 kHz and 22 kHz.

FIG. 26 illustrates the ratio of pixel area of c-Fos staining for the tonotopic domain compared to the average of the areas immediately above and below following a 16 kHz or 22 kHz tone sub-seizure threshold (80 dB) tone stimulation. Error bars represent standard error of the mean. Statistical significance was determined using one-way ANOVA with Tukey's post-hoc analysis at p<0.05. (*) The Frings mice displayed a significantly higher ratio for pixel area than the CF1 and C57BL/6J mice at 16 kHz and 22 kHz.

FIG. 27 illustrates the pattern of c-Fos immunoreactive response in mice placed in the stimulating chamber with the speaker disconnected from the wave generator in: genetically audiogenic seizure (AGS)-susceptible; A) Frings mice and B) congenic mice and AGS-resistant; C) CF1 mice and D) C57BL/6J mice. The AGS-resistant strains displayed focused and narrow tonotopic responses which appeared to correspond to the position of the 16 kHz band. The normal hearing, AGS-resistant mice likely responded to a faint background noise which was below the hearing threshold of the hearing impaired, AGS-susceptible mice.

FIG. 28 illustrates the pattern of c-Fos immunoreactive response following a prolonged, sub-audiogenic (AGS) threshold tone stimulation (11 kHz, 60 dB) in: A) genetically AGS-susceptible Frings mice and AGS-resistant; B) SWR/Bm mice and C) C57BL/6J mice. The Frings and SWR/Bm mice displayed mostly diffuse c-Fos immunoreactivity, however, the C57BL/6J mouse displayed tonotopic focused staining in response to the lower intensity stimulus.

FIG. 29 illustrates the pattern of c-Fos immunoreactive response following a prolonged, sub-audiogenic seizure (AGS) threshold tone stimulation (11 kHz, 100 dB) in: genetically AGS-susceptible; A) Frings mouse and B) congenic mouse and AGS-resistant; C) SWR/Bm mouse and D) C57BL/6J mouse. At the higher intensity stimulus, the Frings mouse had an AGS and displays intense c-Fos immunoreactivity in the 11 kHz domain and in the external and dorsal nuclei of the inferior colliculus. The congenic mouse displayed intense and focused c-Fos immunoreactivity in the 11 kHz tonotopic domain. The SWR/Bm mice did not have an AGS, but displays intense c-Fos immunoreactivity in the anterior medial section of the tonotopic domain and the external nucleus of the inferior colliculus. The C57BL/6J mouse displayed a similar pattern observed with low (60 dB) stimulus but with more diffuse staining ventral medially in the inferior colliculus.

FIG. 30 illustrates regional differences in seizure induced c-Fos expression 2 h following an acute audiogenic seizure (AGS), psychomotor-partial seizures, maximal electroconvulsive seizure (MES) and i.v. pentylenetetrazol (PTZ)-induced (clonic) seizure. Numbers correspond to the level of c-Fos immunoreactivity as qualitatively evaluated: 0=none; 1=diffuse; 2=light; 3=moderate; 4=heavy and nd=not determined. The control mice generally displayed no or light c-Fos immunoreactivity in the brain structures evaluated. AGS resulted in moderate to heavy c-Fos immunoreactivity in brainstem and midbrain structures but not in the forebrain structures. The psychomotor-partial seizures and i.v. PTZ-induced seizure resulted in moderate to heavy seizure-induced c-Fos immunoreactivity in forebrain structures. The MES displayed seizure-induced c-Fos immunoreactivity in both forebrain and brainstem structures.

FIG. 31 shows maximal electroconvulsive seizure thresholds (CC3, CC50 and CC97) of female: ( ) Frings (n=34), ( ) SWR/Bm (n=38), ( ) congenic (n=53) and ( ) C57BL/6J mice (n=42). The congenic mice displayed a significantly lower threshold compared to the C57BL/6J mice and the threshold of the Frings mice was significantly lower compared that of the SWR/Bm mice. The C57BL/6J and congenic mice displayed significantly higher thresholds than the Frings and SWR/Bm mice. The level of statistical significance was calculated by Probit analysis using the MINITAB statistical software. Error bars represent the 95% confidence intervals.

FIG. 32 illustrates maximal electroconvulsive seizure thresholds (CC3, CC50 and CC97) of male: ( ) Frings (n=21), ( ) SWR/Bm (n=25), ( ) congenic (n=39) and ( ) C57BL/6J mice (n=56). The congenic mice displayed a significantly lower threshold compared to the C57BL/6J mice. The C57BL/6J and congenic mice displayed significantly higher thresholds than the Frings and SWR/Bm mice. The level of statistical significance was calculated in Probit analysis using the MINITAB statistical software. Error bars represent the 95% confidence intervals.

FIG. 33 shows psychomotor-partial electroconvulsive seizure thresholds (CC3, CC50 and CC97) of female: ( ) Frings (n=31), ( ) SWR/Bm (n=37), ( ) congenic (n=66) and ( ) C57BL/6J mice (n=55). The C57BL/6J mice displayed a significantly higher threshold compared to the congenic, SWR/Bm and Frings mice. The level of statistical significance was calculated in Probit analysis using the MINITAB statistical software. Error bars represent the 95% confidence intervals.

FIG. 34 illustrates psychomotor-partial electroconvulsive seizure thresholds (CC3, CC50 and CC97) of male: ( ) Frings (n=35), ( ) SWR/Bm (n=27), ( ) congenic (n=63) and ( ) C57BL/6J mice (n=68). No significant differences were observed between the mouse strains. Error bars represent the 95% confidence intervals.

FIG. 35 illustrates minimal electroconvulsive seizure thresholds (CC3, CC50 and CC97) of female: ( ) Frings (n=31), ( ) SWR/Bm (n=24), ( ) congenic (n=42) and ( ) C57BL/6J mice (n=37). No significant differences were observed between the mouse strains. Error bars represent the 95% confidence intervals.

FIG. 36 illustrates minimal electroconvulsive seizure thresholds (CC3, CC50 and CC97) of male: ( ) Frings (n=23), ( ) SWR/Bm (n=25), ( ) congenic (n=44) and ( ) C57BL/6J mice (n=53). No significant differences were observed between the mouse strains. Error bars represent the 95% confidence intervals.

FIG. 37 illustrates the ratio of maximal electroconvulsive seizure threshold (ECT)/ minimal ECT of C57BL/6J, congenic, SWR/Bm and Frings mice. The ratio was calculated from the CC50 results in FIGS. 4.1 and 4.2 for maximal ECT and 4.5 and 4.6 for minimal ECT. A decreasing trend in the maximal ECT/minimal ECT ratio was observed, with the C57BL/6J mice and Frings mice displaying the highest and lowest resistance to seizure spread, respectively. With the exception of the SWR/Bm mice, female mice generally displayed lower maximal ECT/minimal ECT ratios than the male mice within each strain. Mice homozygous for the Frings mass1 allele (i.e., Frings and congenic mice) displayed lower maximal ECT/minimal ECT ratios when compared to SWR/Bm mice and C57BL/6J mice.

### DETAILED DESCRIPTION OF THE INVENTION

The present invention relates to DNA for a novel Monogenic Audiogenic Seizure-susceptible gene (mass1). More particularly, the present invention relates to the isolation and characterization of the mouse mass1 gene (SEQ ID NO: 1) and the human mass1 gene (SEQ ID NO: 3). The discovery that the murine mass1 gene is mutated in Frings mice suggests that mass1 has a role in seizure susceptibility. In addition, the invention relates to a hearing impairment associated with the Frings mass1 mutation.

Nucleotide sequences complementary to the nucleotide sequences of SEQ ID NO: 1 and SEQ ID NO: 3 are also provided. Isolated and purified nucleotide sequences that code for the amino acid sequence of the mouse MASS1 (SEQ ID NO: 2) protein are also within the scope of the invention. Nucleotide sequences that code for the amino acid sequence of the human MASS1 (SEQ ID NO: 4) protein are within the scope of the invention. A nucleic acid sequence that codes for MASS1 of any mammal is also within the scope of the invention.

The nucleic acid molecules that code for mammalian MASS1 proteins, such as a human or murine MASS1, can be contained within recombinant vectors such as plasmids, recombinant phages or viruses, transposons, cosmids, or artificial chromosomes. Such vectors can also include elements that control the replication and expression of the mass1 nucleic acid sequences. The vectors can also have sequences that allow for the screening or selection of cells containing the vector. Such screening or selection sequences can include antibiotic resistance genes. The recombinant vectors can be prokaryotic expression vectors or eukaryotic expression vectors. The nucleic acid coding for MASS1 can be linked to a heterologous promoter.

Host cells comprising a nucleic acid that codes for mammalian MASS1 are also provided. The host cells can be prepared by transfecting an appropriate nucleic acid into a cell using transfection techniques that are known in the art. These techniques include calcium phosphate co-precipitation, microinjection, electroporation, liposome-mediated gene transfer, and high velocity microprojectiles.

The invention further provides methods of evaluating the pharmacokinetics, potential therapeutic value, and/or potential medical significance of a proposed anticonvulsant agent. Specifically, according to the invention, these may be evaluated by providing the proposed anticonvulsant agent to a transgenic mammal in which one or both alleles of the endogenous mass1 gene has been mutated, and examining the therapeutic value or medical significance of the proposed anticonvulsant agent in the transgenic mammal. In methods of the invention, the transgenic mammal may be a mouse, or more specifically, may be a Frings mouse. The value and/or significance of the agent may be evaluated by exposing the mammal to an intense auditory stimulation and observing whether or not a seizure is induced. In addition, observations of the intensity, penetration, and spread of a seizure may be useful in determining the efficacy of the proposed agent. Potential agents may raise the seizure threshold of the transgenic mammal and/or may decrease the spread of any seizure in the mammal.

The Frings mouse is unique among rodent epilepsy models. It is a naturally-occurring single gene model of audiogenic generalized seizures without any other associated neurological or behavioral phenotypes. Sequencing of cosmids from the nonrecombinant mass1 interval identified a single gene. Until recently, computer-based BLAST nucleotide sequence similarity searches did not identify significant similarity between the mass1 sequence and any other sequences in the databases. The deficiency of mass1 cDNA sequence in the databases further supports the hypothesis that mass1 is expressed in low abundance in the brain or that it is degraded very rapidly. This hypothesis is based on the fact that screening two independent brain cDNA libraries for the mass1 cDNA did not produce any positive clones, and low message levels were further supported by Northern blots, RT-PCR, and in situ hybridization. The low abundance could be due to low expression of the mass1 mRNA, or to the message being unstable and quickly degraded.

The mass1 gene was identified by positional cloning and sequencing, exon prediction, RT-PCR and PCR-based 5′ and 3′ RACE. Screening several cDNA libraries by hybridization had not identified a mass1 cDNA clone. Despite not finding a cDNA clone in the cDNA libraries, convincing data implicates mass1 as the gene causing AGS in the Frings mice. Mass1 is the only gene found in the small non-recombinant mass1 interval. The cDNA from both mouse and human Marathon cDNA libraries (Clontech, Palo Alto, Calif.) can be amplified. The intron-exon boundaries are conserved for the genomic structure of hMass1. The alternate transcript of mouse mass1 exon 27 is also found in hmass1. The mass1 transcripts contain long open reading frames which are disrupted by a single base-pair deletion in the Frings mouse.

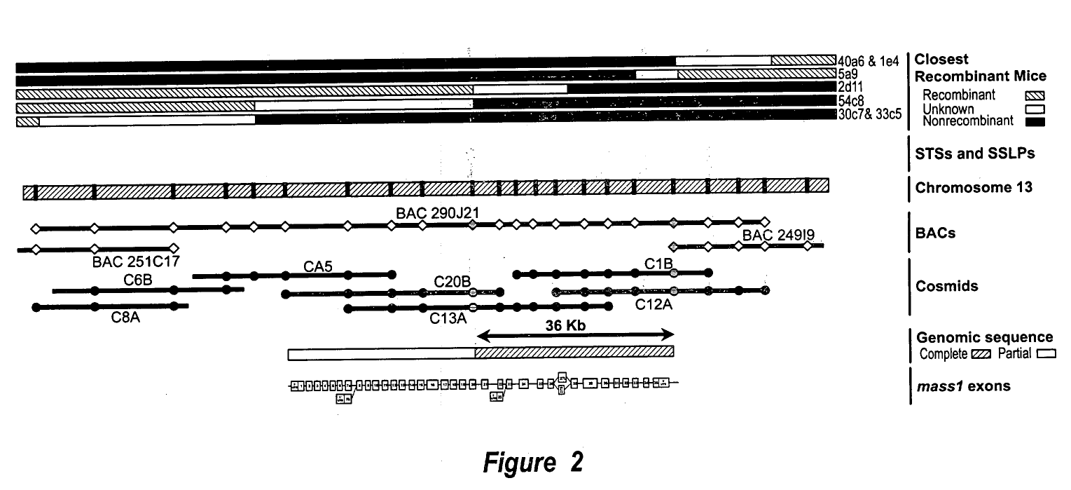

PCR approaches have been required to clone all or parts of other genes such as the melatonin receptor. Reppert, S. M. et al. (1994), Neuron 13: 1177-85. In such cases, results must be viewed with caution because of artifacts inherent with PCR-based assays. Problems include producing inaccurate sequence due to Taq DNA polymerase errors and errors due to amplifying parts of homologous genes. To avoid these problems, the mass1 final sequence was compiled from segments amplified with a high fidelity Pfx DNA polymerase (Gibco) to produce accurate sequence from multiple templates. The mass1 cDNA sequence matched exactly with predicted exons from genomic sequencing of cosmids C1B, C13A, and C20B (FIG. 2).

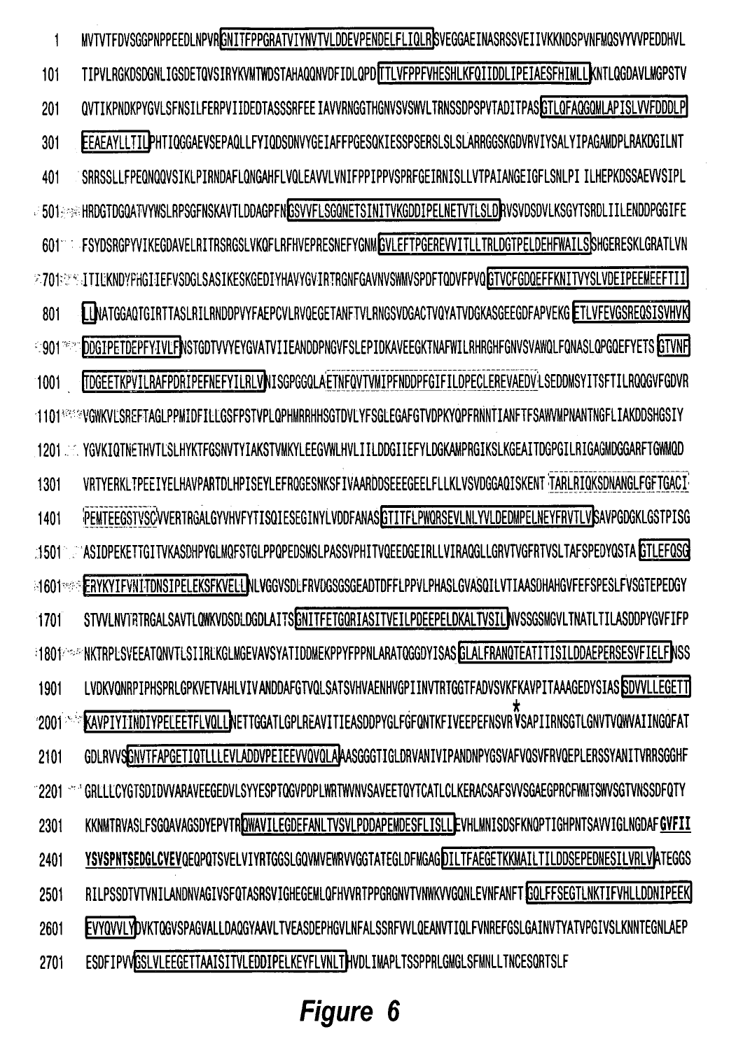

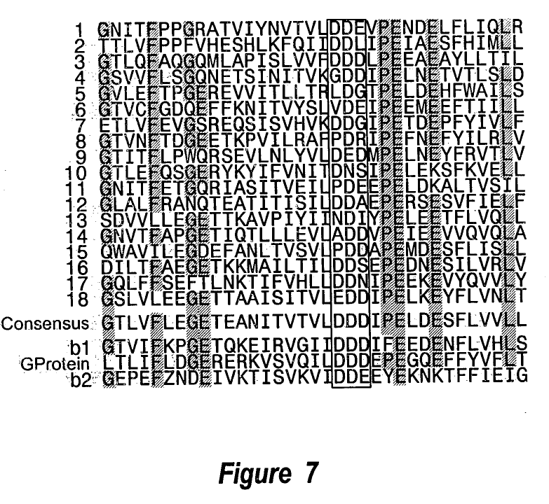

The homology of the MASS1 protein sequence repetitive motifs to the sodium⁺-calcium²⁺ exchanger (Na⁺/Ca²⁺ exchanger) β1 and β2 repeat domains may provide an important clue toward identifying the function of this novel protein. Although the identity between these proteins is limited to a short segment of the cytosolic loop of the exchanger, it is likely to be functionally significant in MASS1 because this motif is repeated 18 times within the protein sequence (FIGS. 6 and 7). The Na⁺/Ca²⁺ exchanger is a plasma membrane associated protein that co-transports three sodium ions into a cell and one calcium ion out of the cell using the sodium electrochemical gradient. Nicoll et al., supra. The Na⁺/Ca²⁺ exchanger can be regulated by intracellular calcium at a Ca²⁺ binding site on the third cytosolic loop that is distinct from the Ca²⁺transport site. This binding site is composed of three aspartate residues (DDD) (FIG. 7). When Ca²⁺is bound at this site, the transporter is activated. Matsuoka, S. et al. (1993), Proc Natl Acad Sci USA 90: 3870-4; Levitsky, D. O. et al. (1994), J Biol Chem 269: 22847-52; Matsuoka, S. et al. (1995), J Gen Physiol 105: 403-20. One of the MASS1 repeats contains the DDD motif, and three others have conservative D to E substitutions suggesting that these domains may be involved in Ca²⁺ binding.

The multicopper oxidase I consensus sequence identified within the MASS1 amino acid sequence is also an interesting putative functional domain. The multicopper oxidases represent a family of proteins that oxidize substrates while reducing molecular O₂ to H₂O. The oxidation of multiple substrate molecules occurs serially while storing electrons in the copper atom (presumably to prevent the formation of reactive species) until a molecule of O₂ is reduced. Two known multicopper oxidases, Fet3p in yeast and ceruloplasmin in humans, have been shown to oxidize and transport iron. Askwith, C. et al. (1994), Cell 76: 403-10; Harris, Z. L. et al. (1995), Proc Natl Acad Sci U S A 92: 2539-43. A third multicopper oxidase, hephaestin has been suggested to be a feroxidase. Vulpe, C. D. et al. (1999), Nat Genet 21: 195-9. Other known multicopper oxidase substrates include Mn²⁺, serotonin, epinephrine, dopamine, and (+)-lysergic acid diethylamide (LSD). Zaitsev, V. N. et al. (1999), J Biol Inorg Chem 4: 579-87; Brouwers, G. J. et al. (1999), Appl Environ Microbiol 65: 1762-8. Therefore, loss of this putative functional domain could possibly result in problems with the metabolism of iron or other metals, copper sequestration, neurotransmitter processing, and/or oxidative stress. Furthermore, the tyrosine kinase and cAMP/cGMP dependent phosphorylation sites may be functionally significant. However, with a large protein such as MASS1, similarities and identities to functional domains commonly occur by chance, and detailed biochemical analysis of the protein will be required to determine which of these motifs are functional domains.

The human orthologue of the mass1 gene resides on chromosome 5q. Interestingly, a gene causing a human epilepsy has also been mapped to this region of chromosome 5. This locus, FEB4, was mapped in families with a phenotype of febrile convulsions. Nakayama, J. et al. (2000), Hum Mol Genet 9: 87-91. While this temperature-sensitive phenotype is different than audiogenic seizures, hMass1 will be an important candidate to test in the FEB4-linked families.

To date, all genes that have been shown to cause non-symptomatic epilepsies have encoded ion channels (voltage- or ligand-gated and exchangers). Jen & Ptacek, supra; Noebels, supra. The mass1 gene therefore represents the first novel gene shown to cause a non-symptomatic epilepsy. The seizures in the Frings mice are different from those recognized to be caused by ion channels. The phenotype is a reflex epilepsy with seizures in response to loud auditory stimuli. This suggests that the genesis of episodes may be in brainstem rather than being due to hyperexcitability of cortical neurons. There is a growing appreciation of the role that deep brain structures and brainstem play in the integration and modulation of cortical discharges. For example, normal synchronized discharges are seen in EEGs of sleeping individuals. Perhaps some of the reflex epilepsies in humans are not the result of primary cortical hyperexcitability, but rather, of abnormal function of circuits critical for integration and modulation of cortical activity. Much work will be required to test this hypothesis, but some fascinating episodic CNS disorders have clinical and electrical manifestation that may be consistent with this idea. Fouad, G. T. et al. (1996), Am. J Hum. Genet. 59: 135-139; Ptacek, L. J. (1998), Genetics of Focal Epilepsies. P. Genton. London, John Libbey. pp 203-13; Plaster, N. M. et al. (1999), Neurology 53: 1180-3; Swoboda, K. J. et al. (2000), Neurology 55: 224-30.

Identification and characterization of the mass1 gene reveals it to be a novel and rare transcript. Further research to determine the function of MASS1 will lead to understanding of how a defect in this protein results in seizures in these audiogenic seizure-susceptible mice. From the mouse mass1 cDNA, a partial human mass1 homolog has been identified. Through mapping and characterization of the human homolog, it may be possible to find an association of mass1 with a human epilepsy disorder. Together, the studies of the mouse and human MASS1 will provide insight into the function of this novel protein and is likely to lead to new insights into normal neuronal excitability and dysfunction of membrane excitability that can lead to seizures and epilepsy.

The present invention also provides transgenic mice in which one or both alleles of the endogenous mass1 gene are mutated. Such animals are useful for example to further study the physiological effects of this gene or to test potential drug candidates.

Methods for making such transgenic animals are known in the art. See, e.g., Hogan et al., Manipulating the Mouse Embryo: A Laboratory Manual (2d ed. 1994); Hasty et al. (1991), Nature 350:243-246; Mansour et al. (1988), Nature 336:348-352. Briefly, a vector containing the desired mutation is introduced into mouse embryonic stem (ES) cells. In some of these stem cells, the desired mutation may be introduced into the cell's genome by homologous recombination. Stem cells carrying the desired mutation may be identified using selection and/or screening procedures. Such cells are then injected into a blastocyst, which may develop into a chimeric mouse with some of the mouse's cells carrying the desired mutation. A chimeric animal carrying germ cells with the desired mutation may be bred to produce mutant offspring.

Vectors containing a desired mutation may be produced using methods known in the art. See, e.g., 1-3 Sambrook et al., Molecular Cloning: A Laboratory Manual (2d ed. 1989). Such vectors would typically include a portion of the mouse mass1 gene to facilitate homologous recombination between the vector and endogenous gene sequences. A selectable marker may be used to disrupt the coding sequence or an expression control element of the mass1 gene. Suitable selectable markers are known in the art. For example, the Neomycin resistance gene (neo), which encodes Aminoglycoside phosphotranferase (APH), allows selection in mammalian cells by conferring resistance to G418 (available from Sigma, St. Louis, Mo.). Other suitable markers may also be used to disrupt the mass1 gene. Techniques have also been developed to introduce more subtle mutations into genes. See, e.g., Hasty et al., supra.

Vectors may also include sequences to facilitate selection or screening of ES cells in which the desired mutation has been introduced by homologous recombination. For example, a vector may include one or more copies of a gene such as the herpes simplex virus thymidine kinase gene (HSV-tk) upstream and/or downstream of the mass1 gene sequences. As illustrated in Mansour et al., supra, random integration events would lead to incorporation of the HSV-tk gene into the ES cell genome, while homologous recombination events do not. ES cells carrying randomly integrated vectors (and, therefore, HSV-tk), may be selected against by growing the cells in a medium supplemented with gancyclovir.

A vector containing the desired mutation may be introduced into ES cells in any of a number of ways. For example, electroporation may be used. See Mansour et al., supra. Other techniques for introducing vectors into cells are known in the art, including viral infection, calcium phosphate co-precipitation, direct micro-injection into cultured cells, liposome mediated gene transfer, lipid-mediated transfection, and nucleic acid delivery using high-velocity microprojectiles. Graham et al. (1973), Virol. 52:456-467; M.R. Capecchi (1980), Cell 22:479-488; Mannino et al. (1988), BioTechniques 6:682-690; Felgner et al. (1987), Proc. Natl. Acad. Sci. USA 84:7413-7417; Klein et al. (1987), Nature 327:70-73.

Techniques for preparing, manipulating, and culturing ES cells have been described. See, e.g., Hogan et al., supra; Mansour et al., supra. ES cells carrying the desired mutation may be identified by screening or selection methods that are known in the art, including growth in selective media and screening using PCR-based or DNA hybridization (Southern blotting) techniques.

The present invention further characterizes a moderate hearing impairment associated with the Frings mass1 mutation. Measuring ABR thresholds revealed that Frings mice develop a moderate, very early onset hearing impairment. The hearing threshold at each acoustic stimulus does not increase more than 10-15 dB until advanced age (PND 580), therefore the hearing impairment appears to be stable with a very slow progression. The progression is similar to that observed for CBA mice, which are recognized as normal hearing, with a slow and relatively flat hearing loss similar to that in humans (Zheng et al., 1999).

The Jackson Laboratory in their screening of 80 inbred strains and sub-strains of mice developed a scale to classify hearing impairment as measured by ABR (Zheng et al., 1999). The mean ABR thresholds for the 60 strains and sub-strains of mice with normal hearing was 38 dB for the click stimulus, 29 dB for the 8 kHz tone, 18 dB for 16 kHz tone and 44 dB SPL for 32 kHz tone. Three levels of hearing impairment were defined as: mild impairment (20-40 dB above mean); moderate impairment (41-60 dB above mean); and severe impairment (greater than 60 dB above mean). Based on this scale, with the click and 16 kHz stimuli, the Frings mice are on the boarder of mild to moderately hearing impaired through most of their life, and even at advanced age they remain only moderately hearing impaired. Using these classifications for 10 kHz and 22 kHz, compared to the SWR/Bm and C57BL/6J mice, the Frings mice would still be in the mild to moderate hearing impairment range.

ABR thresholds that were observed for the C57BL/6J mice and SWR/Bm mice in this study were similar to those observed the C57BL/6J mice and SWR/J mice by Zheng et al. (1999). Both of these strains displayed normal early-life ABR thresholds. However, C57BL/6J mice displayed a progressive hearing loss starting at the higher frequencies at PND 84, demonstrating the presence of late-onset hearing loss gene(s) previously reported (Zheng et al., 1999). The SWR/Bm mice possess 4 of the 6 Frings polymorphisms in the mass1 gene that result in amino acid changes, but not the deletion. (Skradski. et al., 2001), demonstrating that the mass1 deletion is probably the mutation contributing to the very early-life hearing impairment. The SWR/Bm mice used in these studies were found to be heterozygous for the Frings mass1 allele. The normal ABR thresholds in the SWR/Bm mice demonstrate that the auditory deficits associated with the Frings mass1 deletion are recessive requiring both Frings alleles for the hearing loss phenotype.

The involvement of the Frings mass1 deletion in the hearing-impaired phenotype is demonstrated by the BUB/bnJ and congenic strains. The BUB/bnJ mice are from similar genetic stock as the Frings mice (Skradski et al., 2001) and are also homozygous for the deletion in the mass1 gene. The BUB/bnJ mice display a moderate, very early-onset hearing impairment similar to the Frings mice. However, unlike the Frings mice, the hearing impairment in the BUB/bnJ mice begins to progress between PND 18 and PND 30. The hearing impairment in the BUB/bnJ mice continues to progress until they are severely hearing impaired at each stimulus tested by PND 330. When the Frings mass1 alleles were placed on the C57BL/6J background, the resulting congenic strain also developed a moderate, very early-onset hearing impairment similar to the Frings mice. This finding further demonstrates that the Frings mass1 gene deletion is responsible for producing the hearing impairment phenotype. Like the BUB/bnJ mice, the hearing impairment in the congenic strain rapidly progresses during adolescence, and by PND 266 progresses to severe hearing impairment. The early progression of the hearing impairment observed in the BUB/bnJ mice and congenic mice (from the C57BL/6J background strain) is likely the result of interactions between the mass1 gene deletion and the age-related hearing loss gene that is present in these stains (Johnson et al., 1997; Zheng et al., 1999; Johnson et al., 2000). The interaction of these genes probably results in an acceleration of the hearing loss phenotype.

The DBA/2J mice are another AGS-susceptible mouse strain. However, unlike the Frings mice, the DBA/2J mice are polygenic for their AGS phenotype and do not have the Frings mass1 mutations (Skradski et al., 2001). The DBA/2J mice display a mild hearing impairment during early life only at the highest frequency tested. The mild hearing impairment rapidly progresses, even at lower frequencies, to a severe hearing impairment by PND 224.

Elevation in ABR thresholds corresponds with the life-cycle of AGS-susceptibility in the genetically sensitive strains. Each of the mouse strains that were AGS-sensitive displayed a mild or moderate hearing impairment when measured at PND 18 or earlier. AGS-sensitivity in the BUB/bnJ, DBA/2J and congenic strains declines sharply within the first postnatal month, at the time progression of their hearing impairment was observed. The duration of AGS-susceptibility for the DBA/2J and BUB/bnJ mice agrees with previous reports (Schreiber and Schlesinger, 1971; Willott and Lu, 1980; Skradski et al., 2001). Frings mice, which maintain their moderate very early life hearing impairment even to advanced age, remain uniquely AGS-sensitive into adulthood. Therefore, the long duration of AGS-susceptibility in Frings mice reflects the stability of the hearing impairment phenotype. The few Frings mice that lost AGS sensitivity during middle age displayed ABR thresholds similar to the AGS-sensitive Frings mice (data not shown). The factors underlying the loss of AGS-sensitivity in some of the Frings mice are not understood.

The ability to experimentally induce AGS-susceptibility in genetically resistant rodents has been previously reported (Henry, 1967; Norris et al., 1977a; Chen and Willott, 1983; Pierson and Snyder-Keller, 1994). In the work by Henry, C57BL/6J mice noise primed between PND 15 and PND 23 displayed varying levels of AGS-susceptibility when challenged with an electric bell at PND 28. The C57BL/6J mice appeared to be optimally sensitive to noise priming at PND 19 when challenged at PND 28. In the present study, noise priming at PND 18 and PND 19 generated AGS sensitivity in the C57BL/6 mice. However, the development of AGS-susceptibility by noise priming resulted in a lower percentage of mice displaying maximal tonic AGS sensitivity than that observed in the genetic models. Generally, about half of the C57BL/6J mice developed AGS-susceptibility. This was true also for the kanamycin-induced AGS-sensitive C57BL/6J mice. Like the genetic strains, both kanamycin treatment and noise priming produced hearing impairment as measured by ABR in young animals, although, the hearing impairment in the noise-primed animals appears to be transient and limited to higher frequency tone stimuli. The kanamycin-treatment produced a more stable hearing impairment across all the stimuli similar to the Frings mice. However, a third of the kanamycin treated mice failed to develop AGS-sensitivity. Since the kanamycin treatment was received over a long time-course and because mice were not tested prior to PND 19, we cannot be sure if the development of hearing impairment is identical to the Frings mice. The timing of the onset for hearing impairment may be a critical factor in the success of inducing AGS-susceptibility. Overall. the ABR thresholds in the experimentally-induced C57BL/6J mice confirm the importance of the moderate hearing impairment in the development of AGS-sensitivity.

Despite having moderately elevated hearing thresholds, Frings mice displayed significantly higher c-Fos immunoreactive cell counts, and a higher pixel area ratio (staining in the tonotopic domain relative to adjacent areas in the inferior colliculus) compared to non-AGS susceptible mice. Therefore, the c-Fos immunoreactivity in the inferior colliculus of Frings mice displayed a greater intensity that was-focused in the tonotopic response domain. This finding demonstrates that an abnormally high level of neuronal activation occurs in the mid- to high-frequency tonotopic domains from intense, but sub-AGS threshold tone stimulations. The genetically susceptible DBA/2J mice generally displayed greater c-Fos immunoreactive cell counts and a greater ratio for pixel area than the non-susceptible mice. However, the cell counts for the DBA/2J mice were significantly less than the Frings mice. The Frings mice have a more robust maximal AGS phenotype than the DBA/2J mice and the more intense tonotopic c-Fos staining observed with the Frings mice suggests a greater level of neuronal activation in response to the tone stimulus. Because the inferior colliculus is the recognized site of initiation for AGS, the higher level of neuronal activation observed in the tonotopic domain in Frings mice provides one explanation for the development of their robust maximal AGS phenotype.

The AGS-susceptible, noise-primed C57BL/6J mice displayed significantly higher c-Fos immunoreactive cell counts, and a higher ratio of pixel area, compared to the untreated C57BL/6J mice. These results are similar to those observed in the genetically AGS-susceptible mice. This finding demonstrates that an abnormally high level of neuronal activation developed in the 11 kHz tonotopic domain from the intense, but sub-AGS threshold tone stimulation.

The congenic mice displayed a significantly higher ratio for the pixel area of c-Fos immunoreactivity compared to the C57BL/6J background strain. However, the immunoreactive cell counts for the congenic mice were not different from the C57BL/6J mice. These results revealed that congenic mice displayed a more focused tonotopic response, compared to the C57BL/6J, but the intensity was not as high as the other AGS-susceptible strains. The tonotopic mapping experiments were conducted at PND 42, which for congenic mice represents the beginning of rapid progression of their hearing loss. The lower number of c-Fos immunoreactive cell counts observed might be due to the temporal proximity of the tonotopic testing to changes in their hearing thresholds.

The tonotopic response displayed by the AGS-sensitive mice was different than that reported for experimentally induced AGS-susceptible Wistar rats where they observed profound broadening of the tonotopic response domains compared- to normal rats (Pierson and Snyder-Keller, 1994; Kwon and Pierson, 1997). The broader tonotopic responses resembled those of immature rats, suggesting that noise priming arrested the maturation of the tonotopic domains (Pierson and Snyder-Keller, 1994). In the present study, broader tonotopic domains were not observed in either the genetically susceptible or the experimentally induced AGS-susceptible mice, compared to normal mice. This finding suggests that the development of a broader tonotopic response may be species specific or dependent on the developmental age at which the animals were noise primed.

Elevated hearing thresholds in early postnatal development appear to be sufficient for the development of AGS sensitivity, as demonstrated by the experimentally induced AGS-susceptible rodents. A hearing deficit during postnatal development appears to lead to the development of hypersensitivity or hyper-responsiveness of the inferior colliculus as was observed in the tonotopic mapping. However, the hyper-responsiveness leading to AGS-susceptibility was lost if hearing sensitivity rapidly declined due to progressive hearing loss as demonstrated for BUB/bnJ, DBA/2J and congenic mice. Faingold et al. (1990) demonstrated that GEPR 9 rats, which are genetically maximal AGS-sensitive, have a moderate hearing impairment while the GEPR 3 rats, which do not display maximal tonic AGS, are more profoundly hearing-impaired. Furthermore, occlusion of the external ear or tympanic membrane disruption, which can induce developmental AGS-sensitivity in mice, has been shown to increase hearing thresholds about 30 dB (McGinn et al., 1973) placing them in the mild to moderate hearing-impaired category. In the current studies, the noise primed and kanamycin-treated C57BL/6J mice also displayed moderate hearing impairment. Taken together, these results demonstrate that development and maintenance of maximal AGS-sensitivity requires the development of a mild to moderate non-progressive hearing impairment. A severe or rapidly progressive hearing impairment probably causes the development of hyper-responsiveness in the inferior colliculus, but there may not be sufficient afferent input to induce AGS when presented with an acoustic stimulation. The hearing impairment caused by the Frings mass1 gene deletion appears to produce an optimal level of hearing impairment during postnatal development, sufficient to achieve such a very high level of maximal AGS-susceptibility.

There are several hypotheses proposed to explain the relationship between early-life hearing impairment and the development of AGS-sensitivity in affected animals. According to Chen et al. (1983), the factors contributing to the sensitivity of AGS priming are: a period of rapid development in the auditory pathways; the sensitive period of cochlear vulnerability; and the maturational state of inhibitory mechanisms. In C57BL/6J mice at PND 12-13, the external auditory canals are still unopened and neurons in the inferior colliculus are relatively unresponsive to external auditory stimulation (Shnerson and Pujol, 1983). However, by PND 15-17 following hearing onset, neuronal responses in the inferior colliculus develop quickly and begin to resemble those of the adult (Shnerson and Pujol, 1983; Romand and Ehret, 1990). This process continues with tonotopic organization maturing first for the lower frequencies and later for the higher frequencies; the process is not completed until about PND 21 (Shnerson and Pujol, 1983). During this time of rapid maturation, disruption of auditory input would have profound effects on the normal development of the central auditory pathways especially in the inferior colliculus which processes all the auditory information from the brainstem.

The cochlea in young, developing animals is more sensitive to. ototoxicity than it is in adults (Henley and Rybak, 1995), which may also account for the higher success of kanamycin and noise-priming in the developing mice. Without being limited to any one theory, one explanation for the development of AGS-sensitivity, especially from noise priming, suggests that the AGS-sensitivity is due to functional changes in the cochlea that drive. hypersensitivity. Ototoxicity from noise priming and kanamycin results in damage to both the outer and inner sensory hair cells in the cochlear, but preferentially damages the outer hair cells (Chen and Willott, 1983). The inner hair cells are innervated by about 90% of the afferent neurons in the cochlear nerve and account for the bulk of auditory input. The outer hair cells are thought to mechanically modulate the sensitivity of the cochlea and thus the sensitivity of the inner hair cells (Chen and Willott, 1983).

The existence of efferent innervation from brainstem auditory nuclei to the outer hair cells, suggests that this may be an active process that tunes and detunes the cochlea. Therefore, the loss of outer hair cells would result in a loss of sensitivity to lower intensity sounds, but also reduce the ability of the cochlea to desensitize when presented with a loud acoustic stimulation. In animals with more profound outer hair cell damage, an acoustic stimulation such as that during an AGS test would provide greater input to the brainstem auditory structures compared to the normal hearing mice. Furthermore, genetically sensitive AGS models have been shown to have higher outer hair cell loss, including the Rb-1mice, which were derived from Frings stock (Henry and Buzzone, 1986). In fact, the intensity of the tonotopic response in Frings mice to the high intensity tone stimuli might be explained by this model. But one argument against the model of peripherally driven AGS-sensitivity is that transient occlusion or tympanic membrane disruption that does not produce hair cell damage can still produce AGS-susceptibility (for review see Ross and Coleman, 2000). However, the development of AGS-sensitivity is likely to be a more complex process and selective damage to outer hair cells may be one component of the process.

Most of the models of AGS-susceptibility focus on alterations in higher brainstem auditory structures, particularly the inferior colliculus. The fact that after-discharges have been recorded from the higher auditory structures, but not the lower auditory structures of noise-primed C57BL/6J mice suggests that the hyper-responsiveness develops as defects in these structures, and is not simply driven by downstream auditory structures (Chen and Willott, 1983). There is evidence that activity-dependent processes are involved in the maturation of neuronal circuits in brainstem auditory structures (Friauf and Lohmann, 1999; Parks, 1999). An interruption of normal auditory input following hearing onset in mice would interfere with this postnatal activity-dependent maturation which may include effects on neuronal survival, connectivity, neurotransmitter properties and refinement of tonotopic organization (Friauf and Lohmann, 1999; Parks, 1999). In the experimentally induced AGS-susceptible Wistar rats, an immature tonotopic organization was displayed in the inferior colliculus suggesting that refinement of tonotopic organization was arrested (Pierson and Snyder-Keller, 1994). However, the fact that some strains of adult mice, well beyond the critical developmental period, can still be successfully noise primed to develop AGS-susceptibility contradicts this explanation (Chen and Willott, 1983). It may be possible that different mechanisms underlie the development of early postnatal AGS-susceptibility compared to the later life AGS-susceptibility.

Alterations in synaptic properties in the inferior colliculus could also account for the hyperexcitibility leading to the initiation of AGS. Deficits in neurotransmitter systems have been extensively studied in the inferior colliculus of the GEPR (Roberts et al., 1985; Faingold et al., 1986a; Faingold et al., 1986b; Faingold, 2002). Higher levels of glutamatergic activity have been demonstrated in the inferior colliculus of the susceptible rats (Faingold, 1999). An apparent deficit in GABAergic inhibition, despite higher levels of GABA, has also been demonstrated (Faingold, 2002). In normal rats, paired-pulse inhibition observed in the inferior colliculi was replaced with paired pulse facilitation in the GEPR (Li et al., 1994). Microinjection of a GABA antagonist directly in the inferior colliculi of AGS-resistant rats has been demonstrated produce AGS-susceptibility (Millan et al., 1986). This finding demonstrates that loss of inhibitory modulation in the inferior colliculus is sufficient to produce AGS-susceptibility.

Acoustic deprivation by any of the experimental methods shown to induce AGS-susceptibility might produce a deficit in inhibitory mechanisms and lead to hyperexcitability in central auditory structures. This hyperexcitability may be a compensatory mechanism to increase auditory function in the presence of decreased auditory input (Chen and Willott, 1983). However, when the animal is presented with an intense acoustic stimulation the decreased inhibition allows initiation of the AGS. Therefore passive auditory deprivation such as simple occlusion of the external ear could result in development of hypersensitivity of brainstem auditory structures even in adult animals. In the current study, the higher level of neuronal activity in the tonotopic domain in AGS-sensitive mice, demonstrated by c-Fos immunoreactivity, appears to be consistent with a loss of inhibitory input to these neurons.

Sound-induced neuronal responses in the inferior colliculus demonstrated that a significant hyper-responsiveness within tonotopic bands develops in Frings mice compared to AGS-resistant mice. Greater neuronal activation was also observed in tonotopic domains of the DBA/2J and noise primed C57BL/6J mice compared to AGS-resistant mice. Development of AGS-susceptibility in these mice may result from a combined loss of the ability to desensitize the cochlea in the presence of noxious acoustic stimuli: and a hypersensitivity that develops in the higher auditory brainstem structures. The significantly higher level of c-Fos staining observed in the Frings mice, compared to the other AGS-sensitive and AGS-resistant mice, suggest that the development of this severe central hypersensitivity is important in achieving a robust AGS phenotype.

Behavioral electroconvulsive seizure threshold (ECT) testing was used to measure regional neuroexcitability by determining maximal ECT (brainstem), minimal ECT (forebrain) and psychomotor-partial ECT (limbic structures). The objective of these studies was to determine if the Frings mass1 gene deletion affected intrinsic neuroexcitability in the CNS.

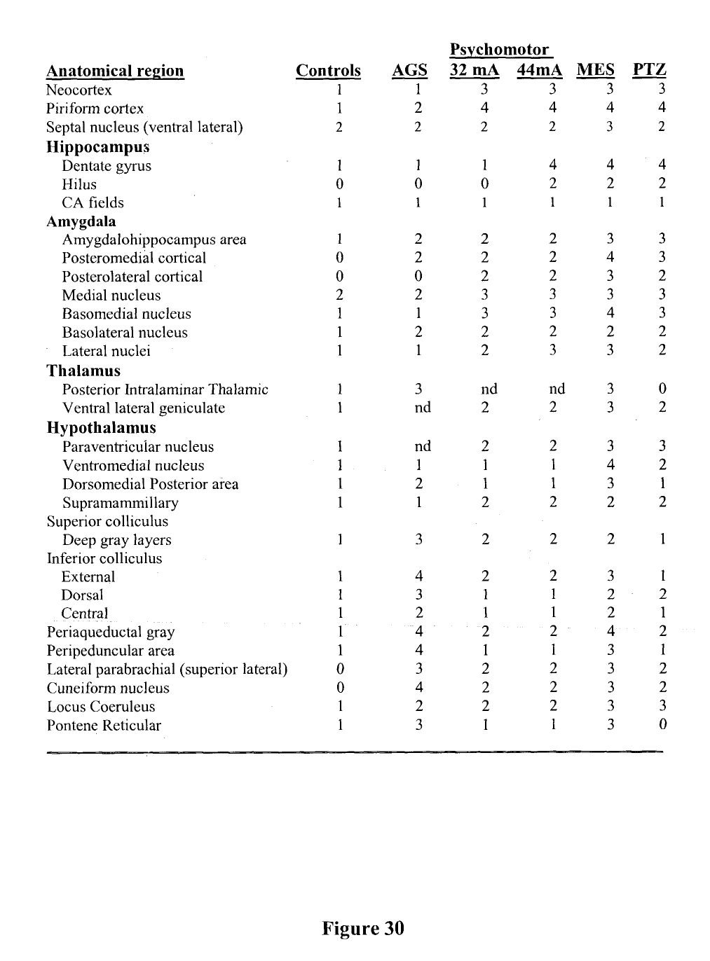

The Frings mice and congenic mice which are homozygous for the Frings mass1 deletion had significantly lower thresholds for maximal electroconvulsive seizures, compared to the SWR/Bm and C57BL/6J mice. These results suggest an effect on brainstem neuronal hyperexcitability associated with the mass1 deletion. The only evidence for lowered threshold in the forebrain was observed in the congenic female mice in the psychomotor-partial (limbic) ECT test. The decreased ratio observed for maximal ECT/minimal ECT observed in the Frings and congenic mice indicates a greater propensity for seizure spread in the mice homozygous for the Frings mass1 allele. The significant effect of the mass1 deletion on maximal ECT corresponds to the evidence that AGS involves a brainstem seizure network (FIG. 30) that does not require input from forebrain structures (Faingold, 1987; Faingold, 1999; Ross and Coleman, 2000).

In order to better describe the details of the present invention, the following discussion is divided into sections: (1) fine mapping and physical mapping of mass1; (2) candidate gene identification; (3) cloning and analysis of mass1 cDNA; (4) mapping of the hMass1 gene; (5) identification of a mass1 mutation in DNA from Frings mice; (6) analysis of the mass1 translated protein sequence; (7) auditory brainstem response thresholds of genetically, audiogenic seizure-susceptible and seizure-resistant mouse strains; (8) audiogenic seizures in genetically-susceptible strains; (9) audiogenic seizure-susceptibility and auditory brainstem response thresholds in experimentally-induced C57BL/6J mice; (10) tonotopic mapping; and (11) behavioral electroconvulsive seizure threshold (ECT) testing.

1. Fine Mapping & Physical Mapping

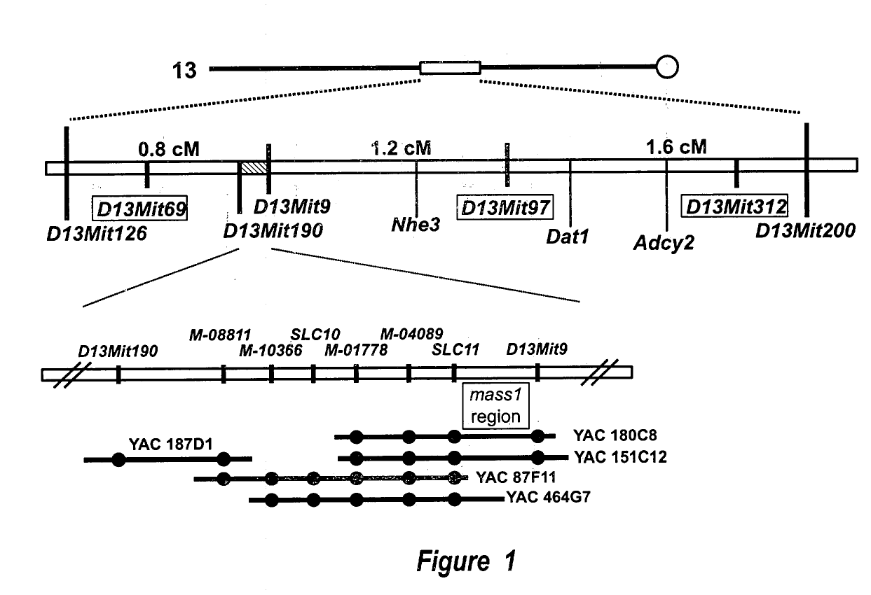

Referring to FIG. 1, the mass1 interval between D13Mit200 to D13Mit126 was estimated to be 3.6 cM with the initial set of 257 N2 mice tested. Skradski, S. L. et al. (1998), Genomics 49: 188-92. Approximately 1200 additional (Frings X C57BL/6J )F1 intercross mice were genotyped with microsatellite markers D13Mit312, D13Mit97, and D13Mit69 that span the interval. Analysis of the recombinations determined that the mass1 region was distal to the D13Mit97 marker and proximal of D13Mit69. Two additional microsatellite markers, D13Mit9 and D13Mit190, were identified within this interval from the Chromosome 13 Committee map. Genotyping of the border-defining recombinant mice with these markers narrowed the interval to between D13Mit9 and D13Mit190. Of the 1200 F2 mice, three were recombinant at D13Mit9 and ten mice were recombinant at D13Mit190. No other known simple sequence length polymorphisms (SSLPs) markers were mapped within this interval.

This distance between the markers D13Mit9 and D13Mit190 was covered by three overlapping YACs 151C12, 87F11, and 187D1 found on the contig WC13.27. These YACs contained four known sequence-tagged sites (STSs), SLC106, SLC117, SLC111 and SLC105 shown in FIG. 2. The four STSs were used to identify BACs from the BAC library. A new single nucleotide polymorphisms was screened by sequencing small-insert pUC19 subclone libraries of the BACs. Two newly identified polymorphic markers, SLC10 and SLC11, were identified and further narrowed the distal border and defined the mass1 interval to the distance spanned by a single YAC, 151C12, between markers SLC11 and D13Mit9 as shown in FIG. 1.

Since no known SSLPs or STSs were contained within the mass1 interval, a physical map of the region was constructed by using end sequences of BAC clones to develop new STSs to re-screen the library for overlapping BACs. Simultaneous with the physical mapping, identification of SSLPs from the new BACs continued to narrow the interval. Seven overlapping BACs were required to cover the distance between SLC11 and D13Mit9. SSLPs from each end of the insert of BAC 290J21, SLC14 and SLC15, were recombinant and localized the mass1 gene to this small region as shown in FIG. 2. Based on the insert size of the BAC, this narrowed the mass1 region to less than 150 Kb.

This BAC insert was subcloned into both a cosmid vector and pUC19. Sequences from randomly selected pUC19 clones were used to develop new STSs across the BAC, and these new markers were then used to align cosmids into a complete contiguous map of BAC 290J21 as shown in FIG. 2. SSLP screening of the pUC19 library detected five new repeat markers within BAC 290J21 (SLC16-20). Two of these, SLC19 and SLC20, were mapped within the mass1 interval. Analysis of recombinants at these markers showed a recombination with SLC20 that refined the interval to two overlapping cosmids, C1B and C13A, between the markers SLC14 and SLC20 each with a single recombinant mouse (5a9 and 2d11).

2. Candidate Gene Identification

Intragenic STS markers were developed for known candidate genes (Dat1, Adcy2, and Nhe3) that-mapped to the general region containing mass1. PCR analysis of the STSs showed that none of the YACs, BACs or cosmids comprising the physical map contained these genes. To directly identify candidate genes from the two cosmids, C1B and C13A, mouse brain cDNA libraries were screened by hybridization using cosmid DNA as probe. The library screening experiments were unsuccessful at identifying any candidate cDNAs from the region, therefore, an alternate strategy of shot-gun subcloning and sequencing of cosmids C1B and C20B was employed.

The cosmid sequences were edited and compiled to produce the complete genomic sequence from marker SLC14 to SLC20. The complete nonrecombinant mass1 interval was approximately 36 Kb. Analysis of the sequence by the exon-finding program, Genefinder, predicted one multiple-exon gene spanning the mass1 interval oriented from the distal to proximal end. Reverse transcription-PCR (RT-PCR) with primers spanning putative introns amplified-products of the appropriate sizes from Frings and C57BL/6J total brain RNA. Sequence analysis of these bands confirmed that they matched the genomic-sequence within the exons and identified the first intron-exon boundaries.

3. Cloning and Analysis of mass1 cDNA

RT-PCR experiments produced 1 Kb of open reading frame that could be amplified from mouse brain RNA. Subsequently, rapid amplification of cDNA ends (RACE) defined the 3′ end of the gene which contained 330 base pairs of untranslated sequence from the first stop codon to the polyA tail. Multiple 5′ RACE reactions produced the complete cDNA sequence of mass1 and identified three putative alternate transcripts each containing a unique 5′ untranslated sequence. When the cDNA sequence was aligned with 36 Kb of complete genomic sequence from cosmid C1B, 15 exons were noted to correspond to the 3′ end of the cDNA sequence; primers were designed from the remaining 5′ cDNA sequence and used to sequence cosmid C20B. Analysis of this genomic sequence revealed 20 exons as shown in FIG. 2. Thus the longest transcript is composed of 35 exons.

The mass1 gene encodes three putative alternate transcripts. The longest transcript is approximately 9.4 Kb, the second 7.1 Kb, and the shortest 3.7 Kb. Northern blot analyses of mouse RNA failed to produce conclusive data to confirm these transcript sizes and suggested that the transcript levels were very low. However, several autoradiograms with very long exposure times (3-4 weeks) suggested that the 9.4 and 7.1 Kb transcripts are expressed in mouse brain (data not shown). In situ hybridizations using a 3 Kb product from the 3′ end of the cDNA to probe mouse brain did not reveal any signal above background further suggesting the mRNA levels to be very low.

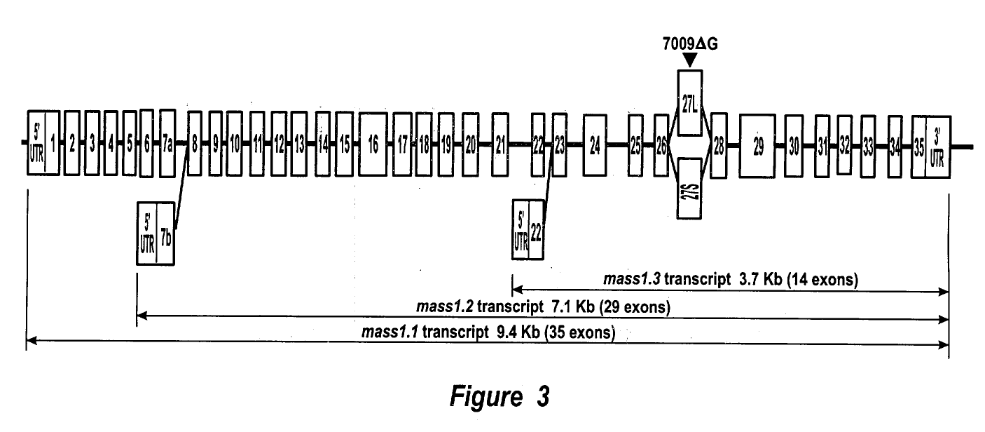

Each putative transcript contains a unique 5′ untranslated region leading into the rest of the gene sequence. All three transcripts contain a possible splice variant in exon 27 where 83 base pairs of sequence are either included (27L) or removed (27S) from the transcript as illustrated in FIG. 3.

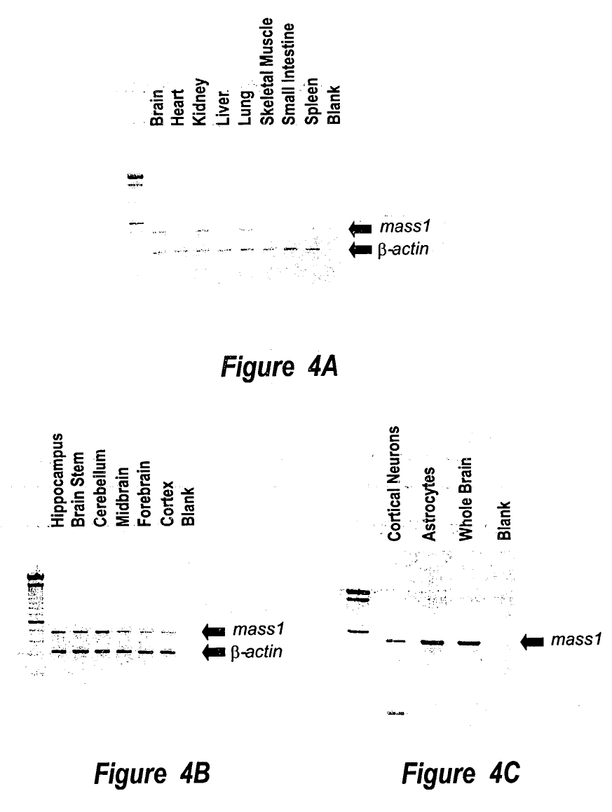

Referring to FIG. 4A, analysis of the expression of mass1 in mouse tissues by RTPCR of brain, heart, kidney, liver, lung, muscle, intestine, and spleen RNA shows that the gene is predominantly found in the brain, lung, and kidney. Further-analysis of the adult mouse brain showed ubiquitous mass1 expression throughout the mouse brain region including hippocampus, brain stem, cerebellum, midbrain and cortex as shown in FIG. 4B. Reverse transcription and PCR revealed mass1 transcripts to be present in RNA isolated from cultured astrocytes and in RNA aspirated and isolated from single mouse cultured cortical neurons as shown in FIG. 4C.

4. Mapping of the hMass1 gene

A human genomic clone containing the human homolog of the mass1 gene was identified by screening a BAC library by PCR with primers from the mouse mass1 gene under lower stringency. This clone was used in fluorescent in situ hybridization experiments and mapped to human chromosome 5q14.

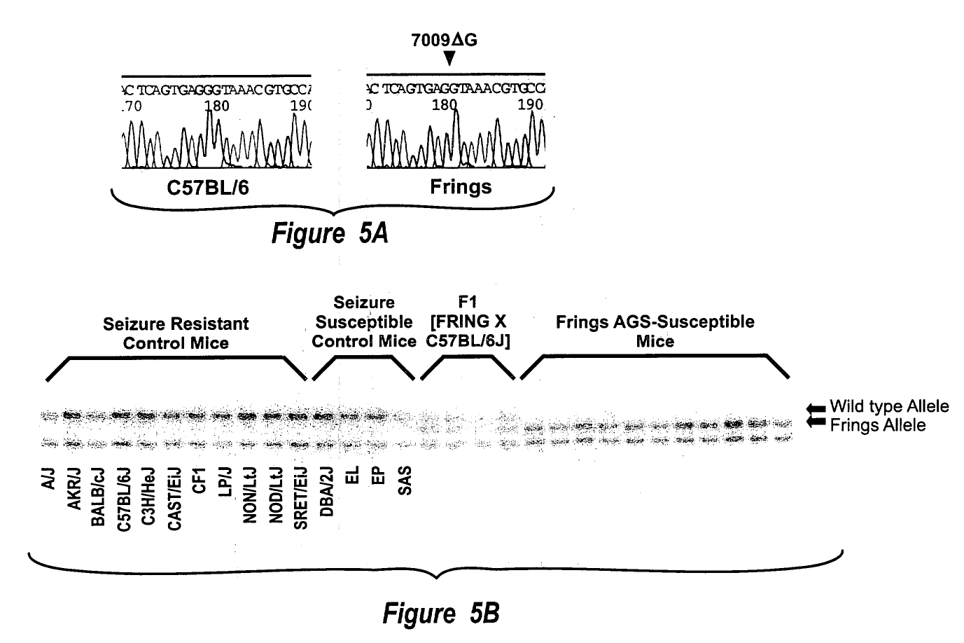

5. Identification of a mass1 mutation in DNA from Frings mice Seventeen single nucleotide polymorphisms (SNPs) were identified between Frings and C57BL/6J mice within the nonrecombinant coding region, exons 21 to 35. One of these SNPs was a single base pair deletion detected in the Frings mouse mass1 gene by sequence analysis of PCR products. FIG. 5A shows the sequence chromatogram of this single G deletion at position 7009 in the Frings mouse DNA sample compared to the seizure-resistant control C57BL/6J. This deletion results in a frame shift of the open reading frame changing the valine to a stop codon; this change is expected to produce a truncated MASS1 protein in Frings mice. Further analysis of the deletion in other mouse strains by gel electrophoresis showed that the deletion is only detected in Frings mouse DNA and not in any of the other seizure-resistant or seizure-susceptible mouse strains tested as shown in FIG. 5b. The deletion is located in exon 27 before the long and short splice variants. Of the other SNPs identified, six altered the amino acid sequence of the protein and could, theoretically, be the genetic basis of Frings audiogenic seizure-susceptibility. Otherwise, these changes represent polymorphisms that may produce subtle alterations in the function of the protein.

6. Analysis of the mass1 Translated Protein Sequence

The mass1 gene produces three putative transcripts: mass1.1 (9.4 Kb), mass1.2 (7.1 Kb), and mass1.3 (3.7 Kb). The long transcript contains 9327 nucleotides and is expected to produce an approximately 337 kilodalton (kD) protein. The medium transcript contains 6714 nucleotides and the predicted protein size is 244 kD. The short transcript open reading frame is 2865 nucleotides and the predicted protein size is approximately 103 kD. These transcripts and isoforms are based on incorporation of the longer splice form of exon 27 (27L). Further putative variants are possible as a result of the 27S alternate splicing event. Using the 27S exon theoretically shortens all the transcripts by 83 nucleotides and each of the isoforms by 645 amino acids (approximately 69.4 kD). The conceptual translation of the amino acid sequence for the mass1.1(27L) transcript is shown in FIG. 6. The MASS1 protein is strongly acidic and has a −192 charge at pH 7.0. The hydropathy plot indicated numerous hydrophobic domains that are candidates for transmembrane segments.

Database searches using the mass1.1 sequence identified no expressed sequence tags (ESTs) that were identical and no homologous genes. However, a small repetitive motif from MASS1 shared homology with numerous Na⁺/Ca²⁺ exchangers. This homology was to the β1 and β2 repeats in the third cytosolic loop of the exchanger that contains the Ca²⁺ regulatory binding domain. Nicoll, D. A. et al. (1996), Ann N Y Acad Sci 779: 86-92. Further analysis of MASSI determined that this motif occurs 18 times within the sequence. Alignment of these sequences shows several highly conserved amino acids within this motif (FIG. 7) including a Proline-Glutamate-X-X-Glutamate (PEXXE) amino acid sequence (SEQ ID NO: 28) that is preceded by one to three acidic residues (D or E). The proline and first glutamate are completely conserved in all 18 related motifs, and the second glutamate is conserved in 16 of the motifs. In repeats 10 and 1, a lysine is substituted for the second glutamate. The PEXXE motif occurs twice more within the MASS1 sequence, however, these repeats (repeats 19 and 20) have a lower degree of identity and similarity (FIG. 6).

Three aspartic acid residues (DDD) are found in the Na+/Ca²⁺exchanger β1 segment and in the segment of the very large G-protein coupled receptor-1 directly preceding the PEXXE motif. In the MASS1 repeat, however, this DDD motif is not well conserved with only repeat number 3 containing the exact DDD motif, and repeats 1, 9, and 18 containing conservative substitutions of glutamate residues. The 18 repeats are distributed across the MASS1 protein and repeats 14 to 18 would be missing from the truncated MASS1 protein (FIG. 6).

Analysis of the MASS1 sequence by Pattern Match identified a multicopper oxidase I consensus sequence site in the carboxyl-terminal region of MASS1. The multicopper oxidase I site is located in exon 29 (FIG. 6), within the region of the MASS 1 protein that would be truncated by the Frings 7009ΔG mutation. Frings mice would therefore be lacking this potentially important domain. Biochemical analysis of this putative domain will determine if this is a functional multicopper oxidase I domain. Other less common motifs found within MASS1 include three tyrosine kinase phosphorylation motifs, two cAMP/cGMP-dependent phosphorylation motifs, and one glycosaminoglycan attachment motif. Finally, numerous common putative protein modification sites were identified including casein kinase II phosphorylation, protein kinase C phosphorylation, N-myristylation, and N-glycosylation sites. Further analysis of the MASS1 protein will be required to determine if any of these consensus sites are functional.

7. Auditory Brainstem Response Thresholds and Audiogenic Seizures

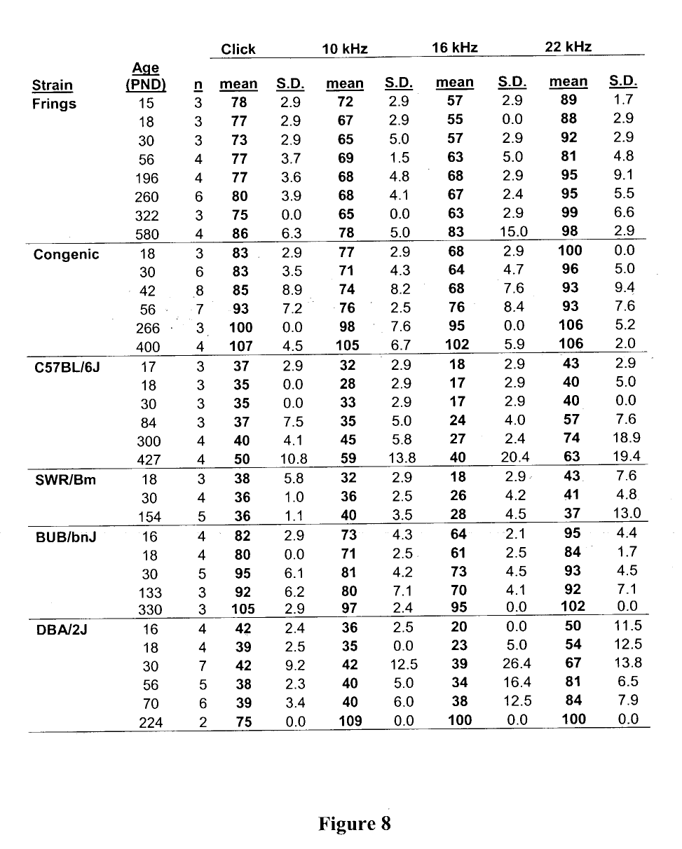

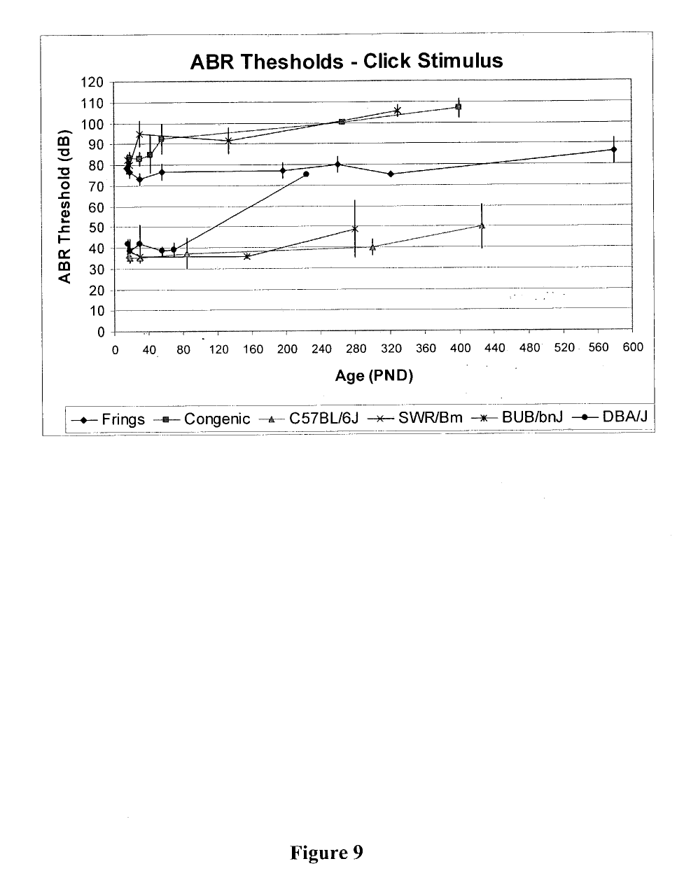

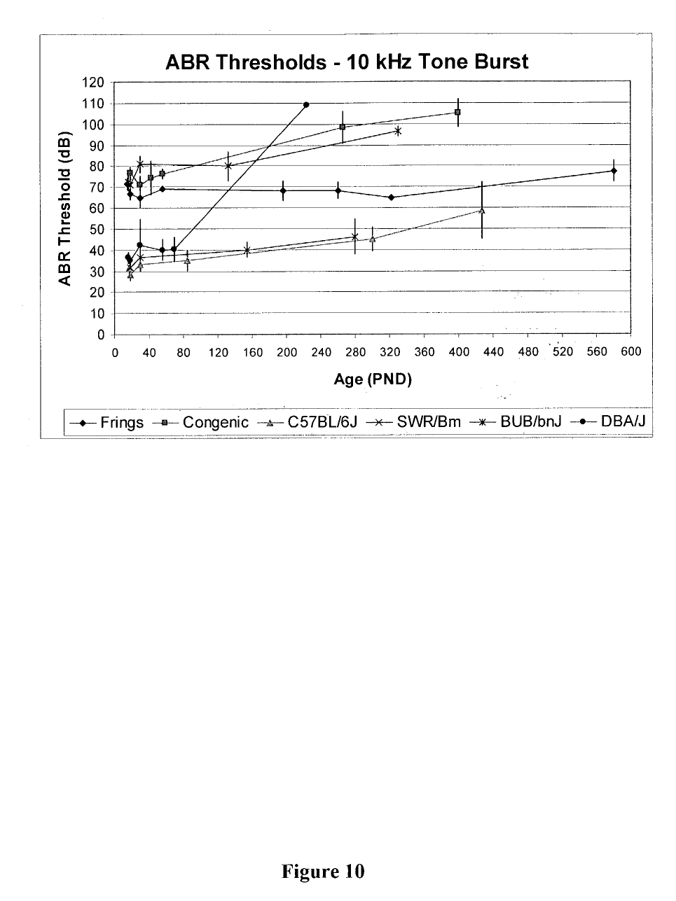

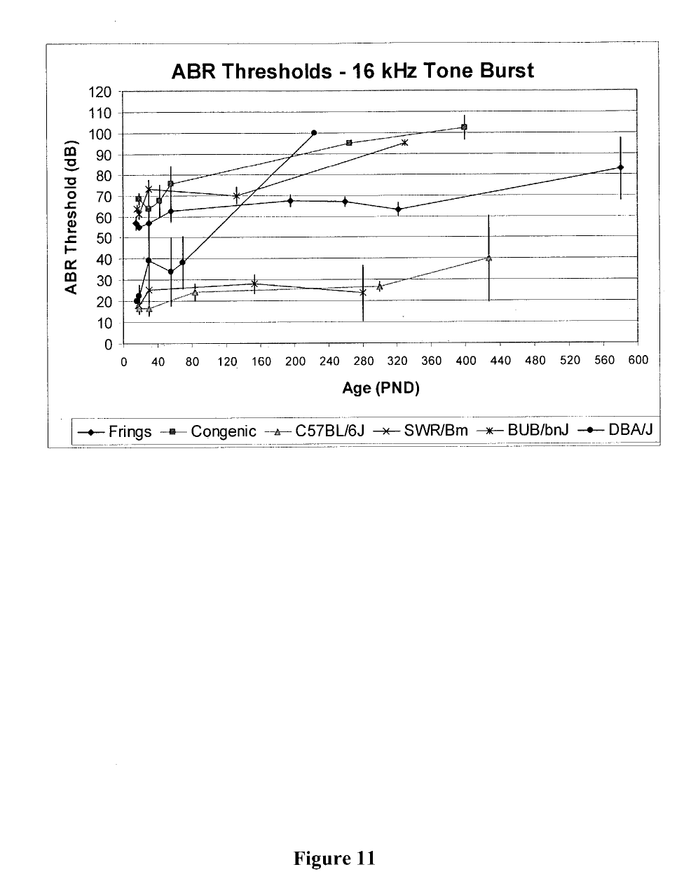

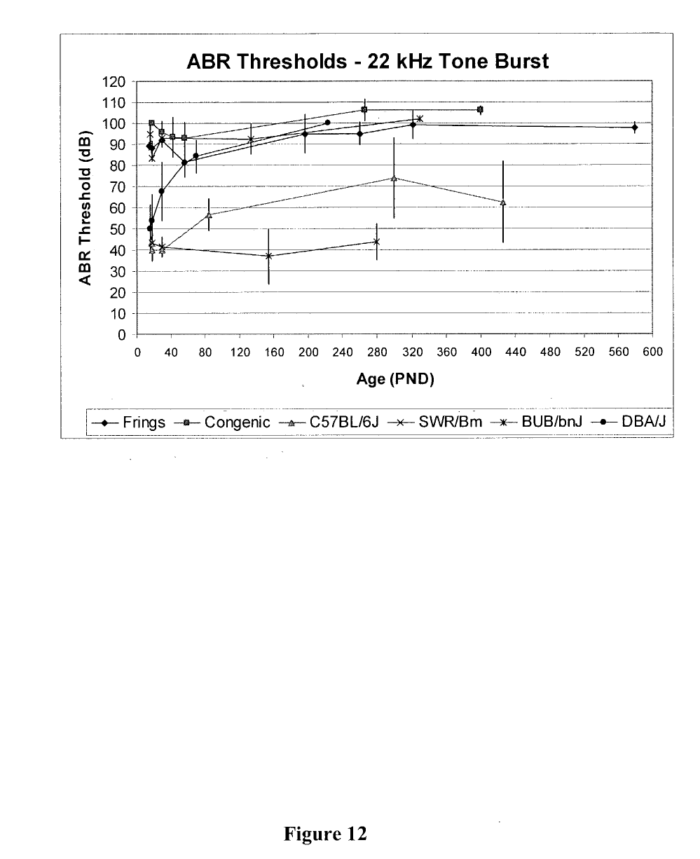

ABR thresholds were determined to assess auditory function in the various AGS sensitive and resistant mouse strains. The average ABR thresholds for each of the acoustic stimuli at various age-points are shown in FIG. 8. In addition, the ABR thresholds are plotted against age for each of the acoustic stimuli; click (FIG. 9), 10 kHz (FIG. 10), 16 kHz (FIG. 11) and 22 kHz (FIG. 12). ABR testing reveals that Frings mice have elevated hearing thresholds as early as PND 15 (the earliest age tested). Frings mice at PND 18 show average ABR thresholds that are elevated approximately 35 dB at each acoustic stimulus compared to the SWR/Bm mice which possess more normal hearing. However, the elevated hearing thresholds for the Frings mice do not progress more than 10 dB for the click and 10 kHz stimuli or 15 dB for the 16 kHz and 22 kHz stimuli until PND 580.

Like the Frings mice, BUB/bnJ mice which possess the Frings mass1 deletion displayed very early elevated ABR thresholds similar to the Frings mice (FIGS. 9-12). Average ABR thresholds at PND 18 for the BUB/bnJ were 35 dB to 44 dB greater than the SWR/Bm mice at each acoustic stimulus tested. However, unlike the Frings mice, the elevated hearing thresholds for BUB/bnJ mice suddenly progress 10 dB or more at each acoustic stimulus between PND 18 and PND 30. The elevated ABR thresholds of the BUB/bnJ mice continue to progress and by PND 330 the thresholds were 95 dB or greater for all acoustic stimuli tested. The C57BL/6J mice displayed normal ABR thresholds until PND 84, then the average ABR threshold at 22 kHz increased 17 dB; elevations of 13 dB to 24 dB were observed at PND 427 for all the stimuli. In contrast, at PND 18 the congenic mice displayed elevated ABR thresholds approximately 50 dB greater than the C57BL/6J mice at each acoustic stimulus. Between PND 30 and PND 56, there was a sudden progression in the average ABR threshold for the click and 16 kHz stimuli of 10 dB and 12 dB respectively. At PND 266 congenic mice displayed ABR thresholds that were 95 dB or greater for all the stimuli evaluated.

The average ABR thresholds of PND 18 DBA/2J mice were similar to SWR/Bm and C57BL/6J mice at all stimuli except the 22 kHz tone where they displayed an elevation of 13 dB to 14 dB greater than the other two strains. Between PND 18 and PND 30 the average ABR threshold for the 16 kHz and 22 kHz stimuli in DBA/2J mice increased by approximately 15 dB. At PND 224 the average ABR thresholds for the DBA/2J mice increased to greater than 95 dB for all but the click stimulus.

8. Audiogenic Seizures in Genetically-Susceptible Strains

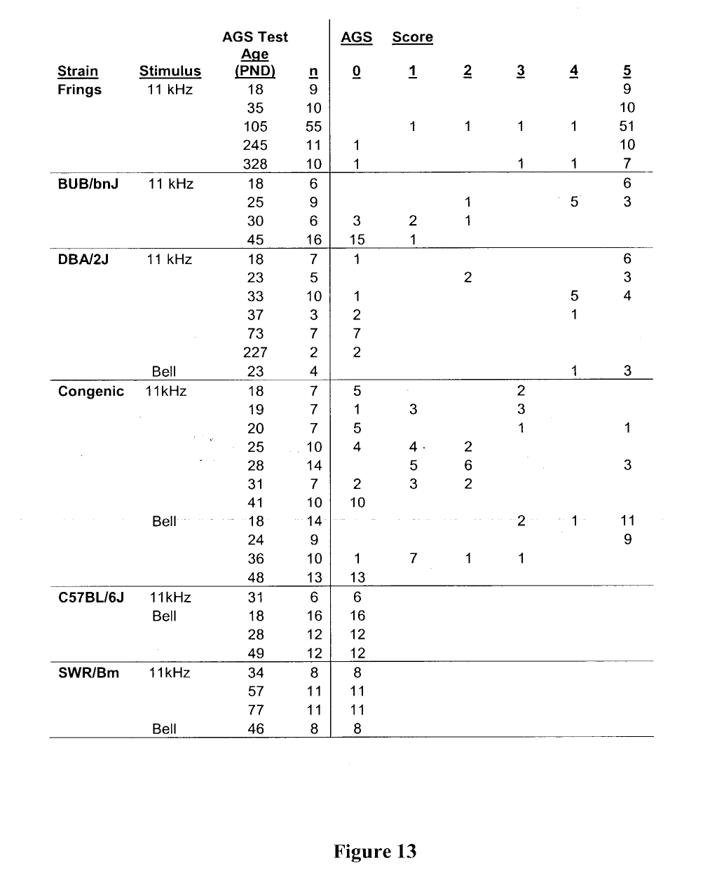

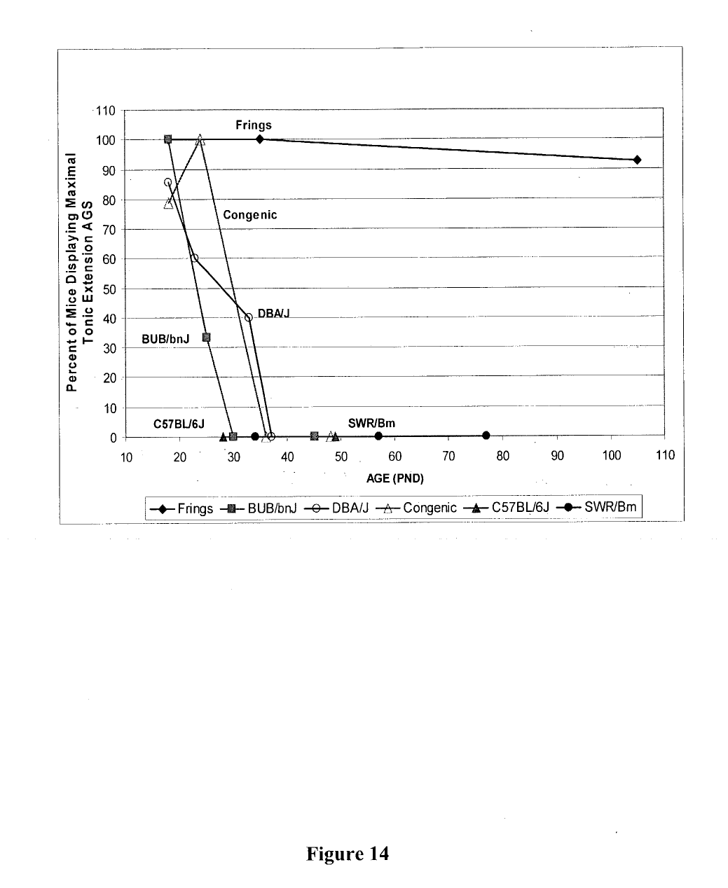

The susceptibility of the different mouse strains to AGS, as measured on the five-point AGS scale (see methods), is shown in FIG. 13. The Frings, BUB/bnJ, DBA/2J and congenic mice all display various degrees of AGS-sensitivity. Susceptibility to maximal tonic AGS as a function of age is plotted in FIG. 14. All of the Frings mice tested at PND 18 and PND 35 displayed maximal AGS when challenged with the 11 kHz tone stimulus. At PND 105 the Frings mice remained highly AGS-sensitive with 92.7% (51/55) displaying maximal tonic AGS. Even at advance ages, PND 245 and PND 378, the Frings mice remained highly AGS-sensitive with 90.9% (10/11) and 70.0% (7/10) displaying tonic AGS respectively.

The SWR/Bm mice were not AGS-susceptible when challenged with the 11 kHz tone or electric bell (FIG. 13). These results demonstrate the AGS-resistance of the SWR/Bm. At PND 18, BUB/bnJ mice displayed 100% (6/6) maximal tonic AGS in response to the 11 kHz stimulus. However, AGS-sensitivity sharply declined at PND 25 for BUB/bnJ mice when only 33.3% (3/9) displayed maximal tonic AGS. At PND 30 the AGS sensitivity of the BUB/bnJ mice declined sharply with only wild running displayed in 50% (3/6) of the mice and the remaining showing no response. By PND 45 only one (1/16) BUB/bnJ mouse displayed any AGS sensitivity, which was wild running only, while the remaining (15/16) showed no response. These results suggest a sudden loss of maximal AGS-sensitivity occurs between PND 18 and PND 25, and a nearly complete loss of AGS-sensitivity by PND 45, in the BUB/bnJ mice.

At PND 18, 85.7% (6/7) of the DBA/2J mice displayed maximal tonic AGS. However, at PND 23 and PND 33 only 60% (3/5) and 40% (4/10), respectively, displayed maximal AGS to the 11 kHz stimulus. By PND 73, the DBA/2J mice no longer displayed AGS-sensitivity in response to the 11 kHz stimulus. These results demonstrated that a steady decline in AGS-sensitivity occurs between PND 18 and PND 73 with the DBA/2J mice.

When challenged with the 11 kHz tone stimulus, congenic mice displayed a very low level of maximal tonic AGS responsiveness (FIG. 13). However, with the electric bell 78.6% (11/14) of the congenic mice tested at PND 18 and 100% (9/9) at PND 24 displayed maximal AGS responses. At PND 36, most of the congenic mice displayed only wild running (7/10) and at PND 48 none of the congenic mice displayed AGS-sensitivity even in response to the electric bell. Like the BUB/bnJ and DBA/2J mice, the congenic mice displayed an early decline in AGS-susceptibility within first postnatal month.

All of the mouse strains that displayed genetic sensitivity to AGS in FIG. 13 also displayed elevated ABR thresholds in very early life. The decline in maximal AGS-sensitivity in BUB/bnJ, DBA/2J and congenic mice (FIG. 14) corresponds to the sudden increase in their elevated ABR thresholds observed after PND 18. In contrast, Frings mice that display relatively stable ABR thresholds maintain a very high level of maximal AGS that persists well beyond that of the other genetically susceptible strains (FIG. 14).

9. Audiogenic Seizure-Susceptibility and Auditory Brainstem Response Thresholds in Experimentally-Induced C57BL/6J Mice

Audiogenic seizures: None of the C

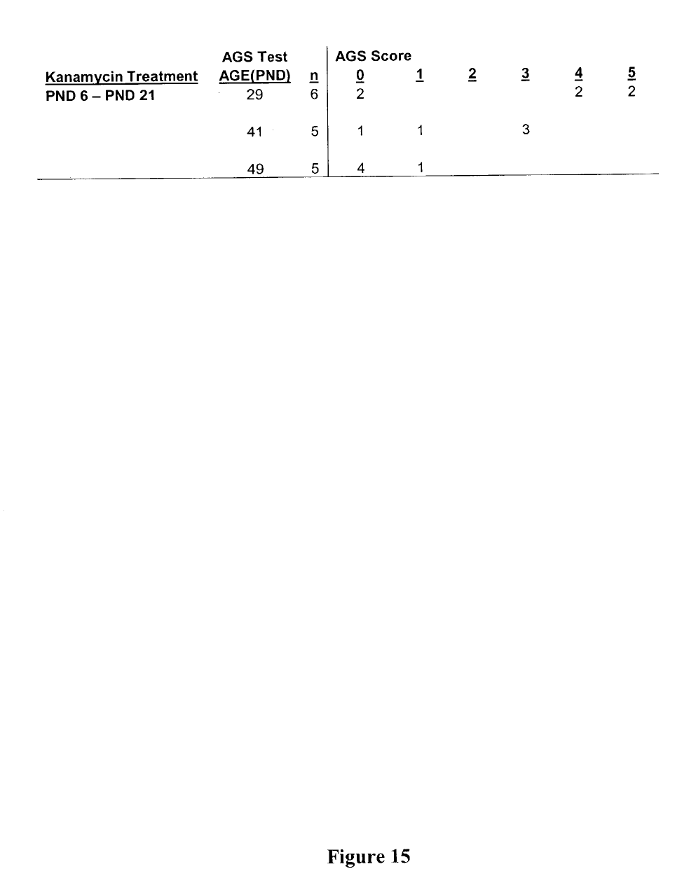

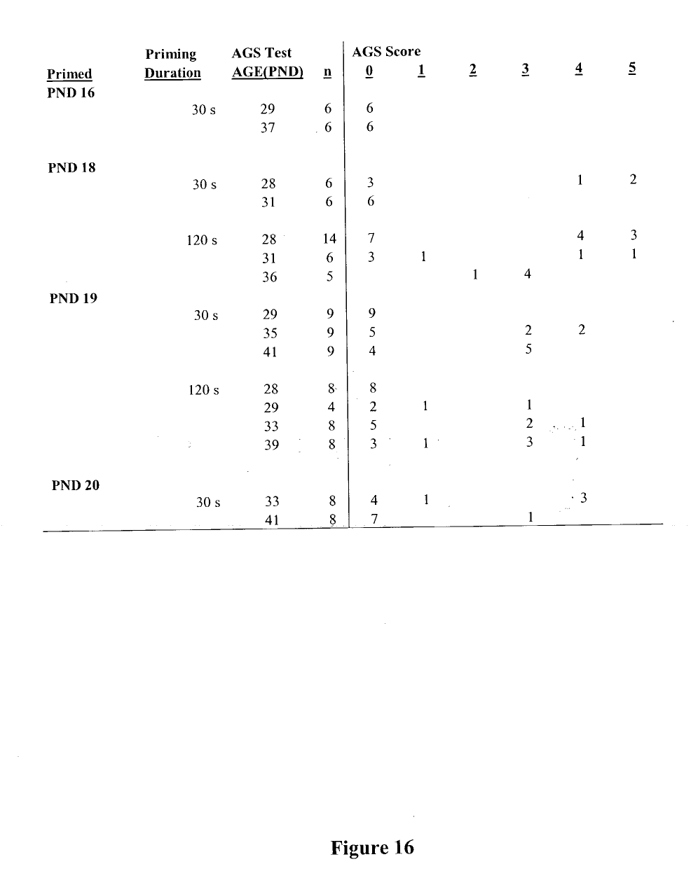

57BL/6J mice were AGS-sensitive when initially challenged with the 11 kHz stimulus or the electric bell (FIG. 13). However, as shown in FIG. 15 and FIG. 16, C57BL/6 mice that were experimentally treated to induce peripheral hearing impairment as pre-weanlings displayed various degrees of AGS-sensitivity. The C57BL/6J mice treated with kanamycin between PND 6 and PND 21, and then challenged with the electric bell at PND 29, displayed development of AGS activity with 66.7% (4/6) having either maximal tonic or clonic AGS (FIG. 15). By PND 49, the kanamycin-induced AGS sensitivity was lost, with only one (1/5) mouse displaying wild running. The saline-treated littermates did not display AGS-sensitivity (4/4—data not shown).

The effectiveness of noise priming the C57BL/6J mice during development was dependent on the age of the mice. None of the mice noise primed at PND 16, PND 19 or PND 20 displayed maximal tonic AGS responses when rechallenged with the electric bell (FIG. 16). Mice noise primed at PND 18 did display maximal tonic AGS sensitivity when challenged at PND 28 (33.3% [2/6] for 30 s and 21.4% [3/14] for 120 s noise exposure). At PND 31, only 16.7% (1/6) of the mice that received a 120 s priming noise exposure displayed maximal tonic AGS, and half (3/6) of the mice displayed no response. The C57BL/6J mice noise primed at PND 19 displayed intermediate AGS scores (stage 3—wild running with loss of righting reflex or stage 4—clonic seizure) when challenged at later ages, between PND 29 and PND 41, but did not display tonic AGS (FIG. 16). These results demonstrate that experimentally induced C57BL/6J mice developed AGS-sensitivity, but the penetrance and AGS severity was generally not as high as that observed in genetically susceptible mice.

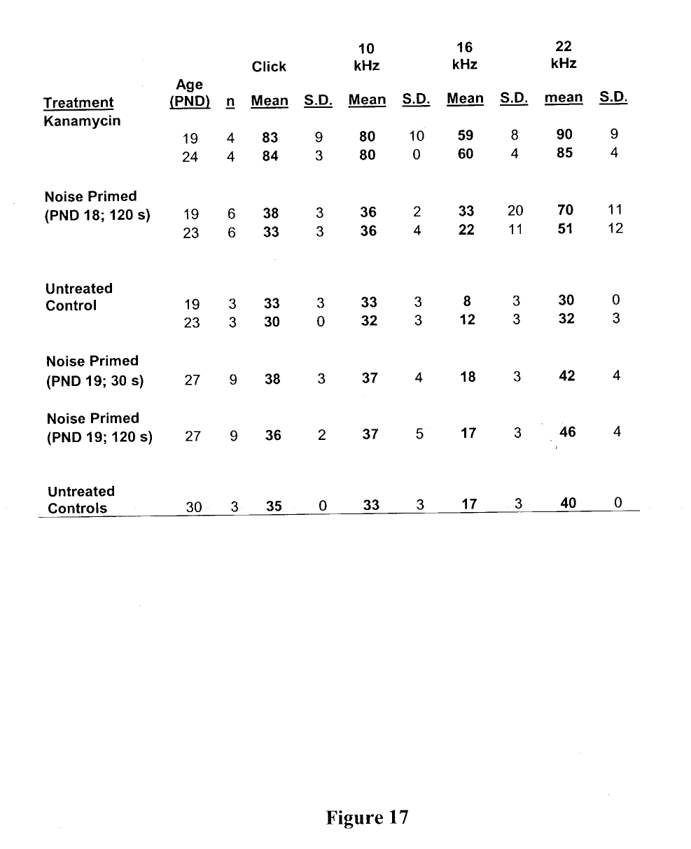

Auditory brainstem response: Like the genetically AGS-sensitive mice, the developmentally induced mice displayed elevated ABR thresholds (FIG. 17). At PND 19 and PND 24, the mean ABR thresholds for the kanamycin-treated mice were stable and very similar to those of the Frings mice for all the acoustic stimuli. C57BL/6J mice noise primed for 120 s at PND 18 and ABR thresholds tested 24 h later revealed elevations of approximately 25 dB at 16 kHz and 40 dB at 22 kHz compared to their non-primed littermates. However, the mean increase in ABR threshold observed on PND 19 had diminished by 50% when the same mice were tested again on PND 23. C57BL/6J mice noise primed at PND 19 for 30 s or 120 s displayed ABR thresholds at PND 27 that were almost identical to age matched C57BL/6J mice. Therefore, the elevations in ABR thresholds resulting from noise-priming appeared to be transient and limited to the 16 kHz and 22 kHz acoustic stimuli.

10. Tonotopic Mapping

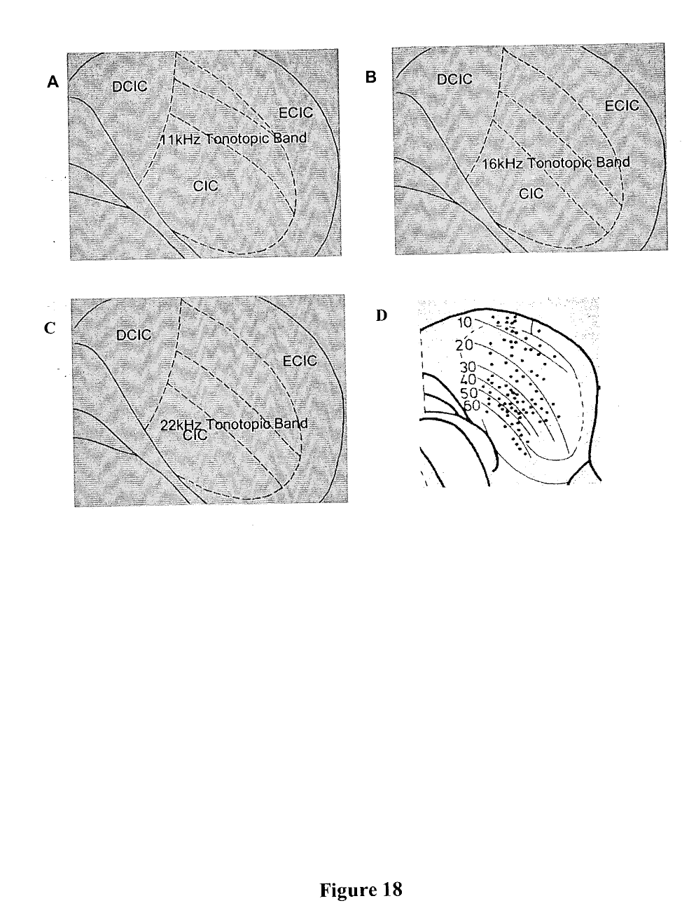

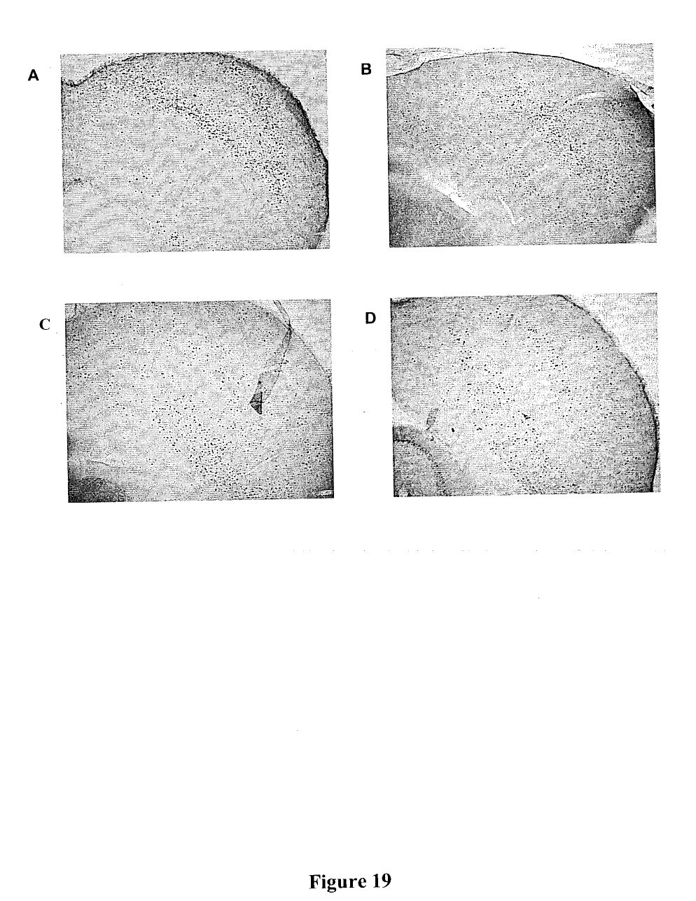

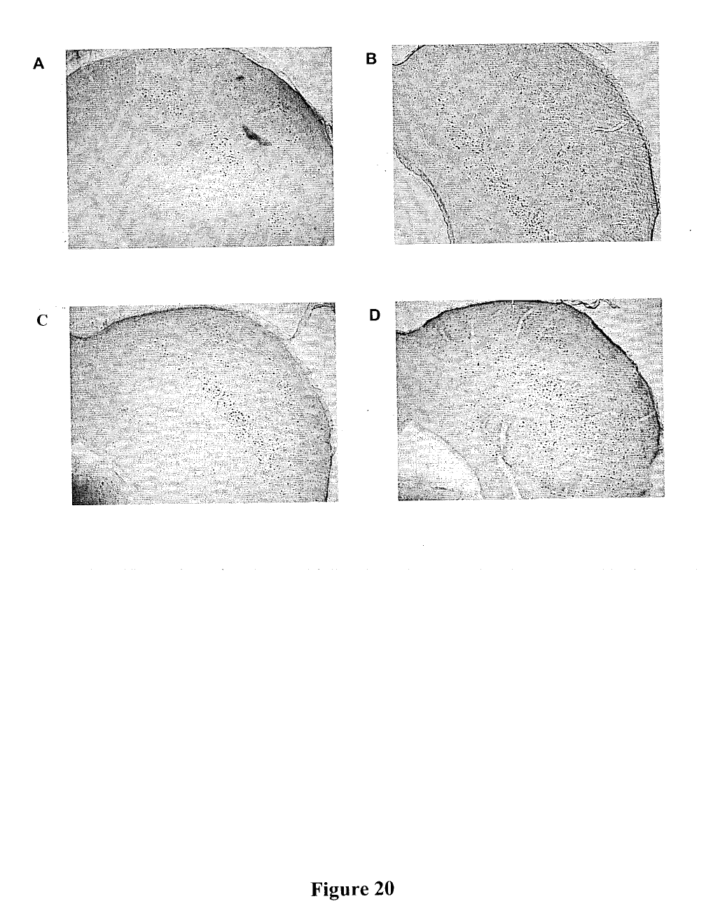

c-Fos positive immunoreactivity was used as marker of neuronal activation in response to prolonged, intense, but sub-AGS threshold tone stimulations. The resulting c-Fos immunoreactive cells, in the frequency domain, were quantified and analyzed for pixel area using a template as shown in FIG. 18. The c-Fos positive tonotopic pattern following the 11 kHz tone stimulation in the AGS-sensitive Frings and DBA/2J mice, compared with the AGS-resistant SWR/Bm and CF1 mice, are displayed in FIG. 19. In FIG. 20, the tonotopic pattern for a genetically AGS-sensitive congenic mouse is displayed against a mouse from the AGS-resistant C57BL/6J parent strain. Also in FIG. 20, a successfully noise primed C57BL/6J mouse is displayed with a C57BL/6J mouse that was noise primed but did not develop AGS-sensitivity.

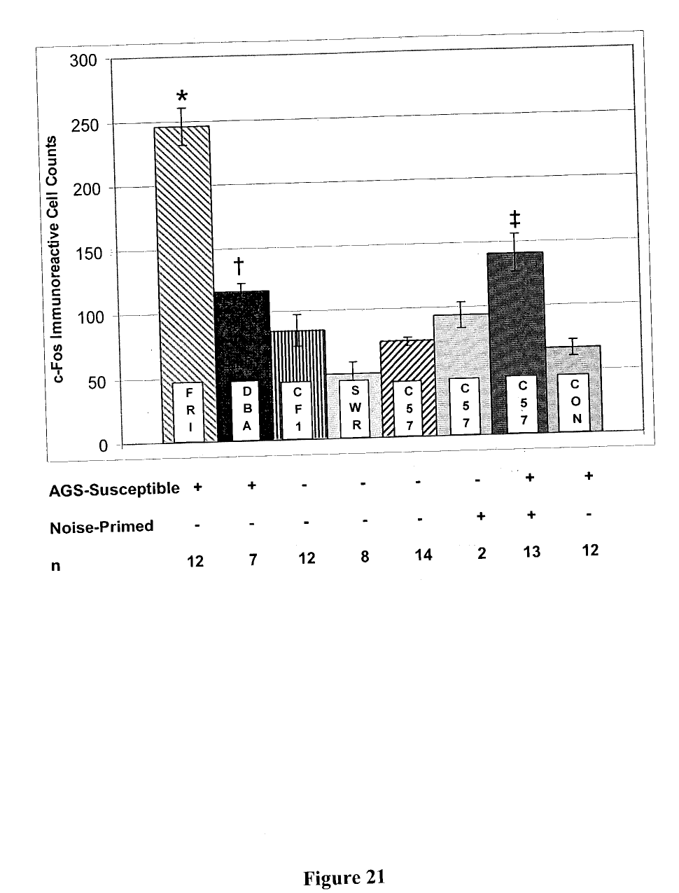

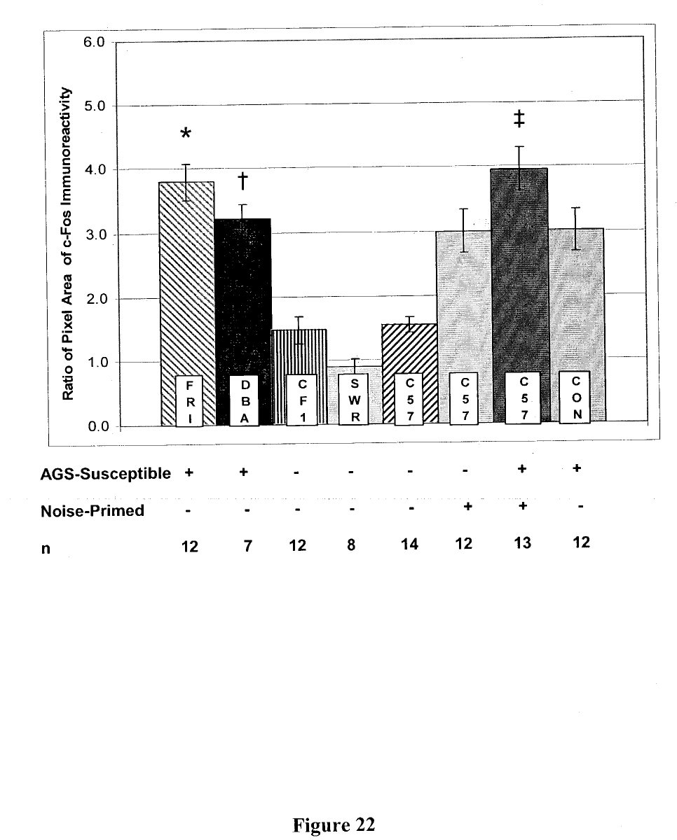

The location of the resulting tonotopic response for each of the tested frequencies correspond to the location determined using electrophysiological techniques in the mouse inferior colliculus (Stiebler and Ehret, 1985). In the AGS-sensitive mice, the c-Fos immunoreactive tonotopic response appears denser, and with less staining than observed in other areas of the central nucleus of the inferior colliculus, compared to the AGS-resistant mice. FIG. 21 shows that Frings mice had a significantly higher number of c-Fos positive cell counts compared to all of the other strains tested, including both the AGS-sensitive and resistant mice. The DBA/2J mice displayed c-Fos positive cell counts that were significantly greater than the AGS-resistant SWR/Bm and C57BL/6J mice, but not significantly greater than the CF1 mice. AGS-susceptibility in C57BL/6J noise-primed mice yielded greater average cell counts compared to noise-primed C57BL/6J that did not develop AGS-sensitivity. The cell counts for the congenic mice were not different from the C57BL/6J parent strain. FIG. 22 shows the mean ratio of the pixel area of c-Fos positive staining following 11 kHz tone stimulus in the tonotopic response domain, compared to the adjacent areas immediately above and below. The trend was similar as that observed in the cell counts. The AGS-sensitive mice displayed a significantly higher average ratio for the pixel area in the tonotopic band, compared to the areas immediately above and below that band (FIG. 22).

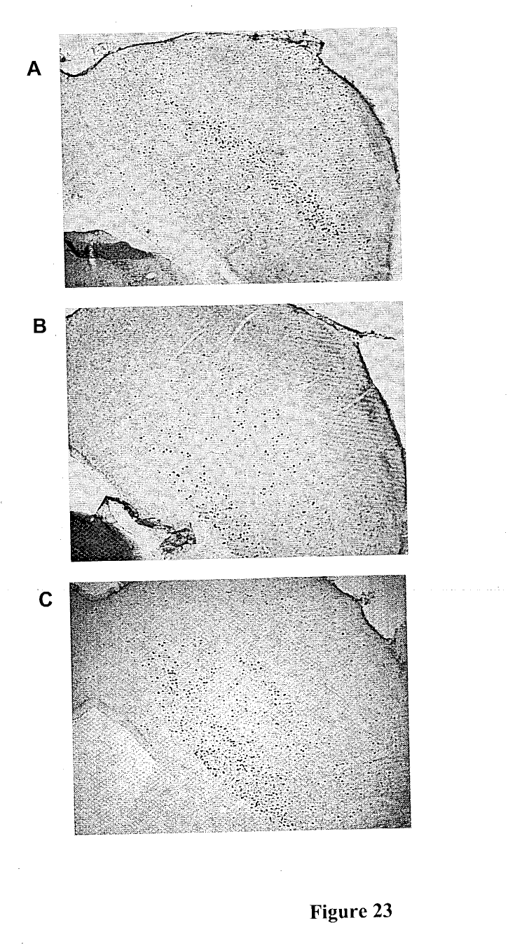

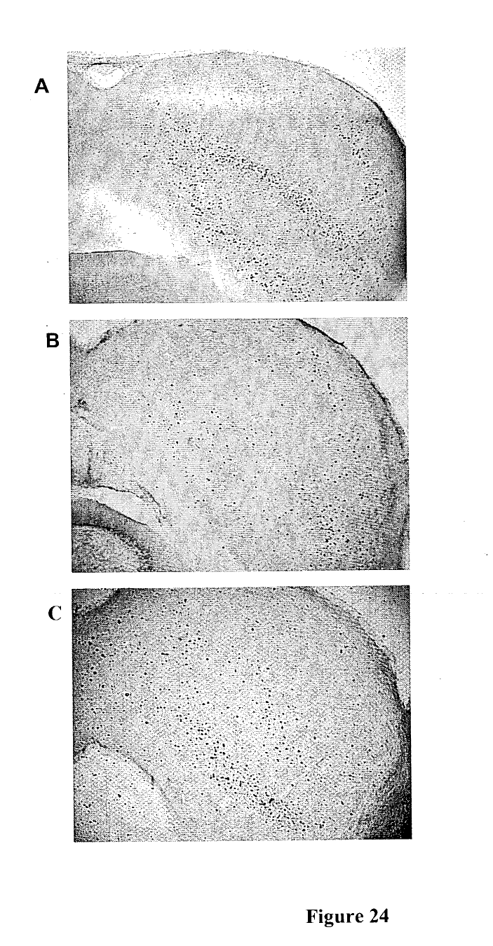

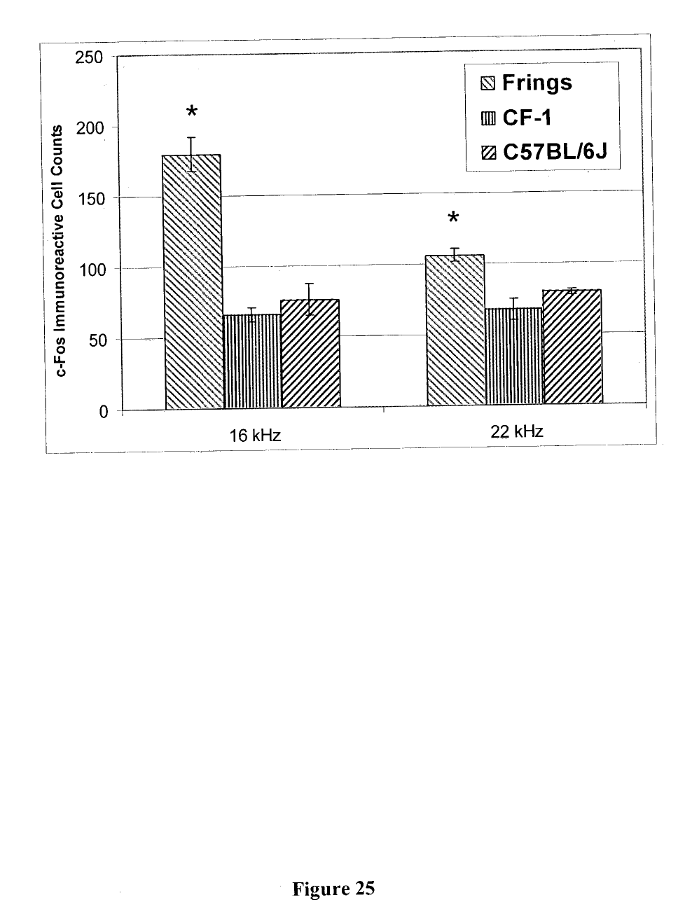

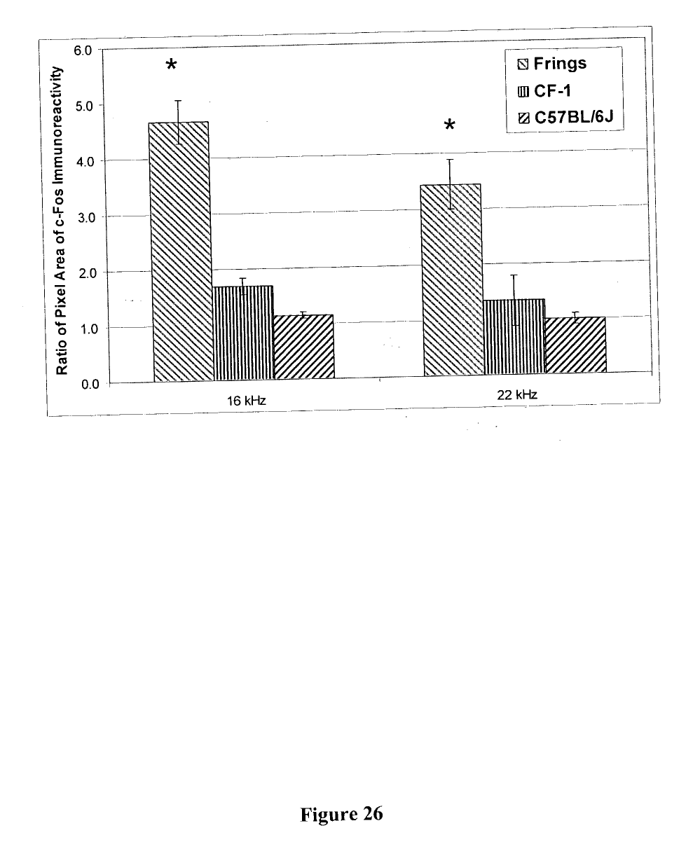

Tonotopic responses were also evaluated in the Frings, CF1 and C57BL/6J mice at 16 kHz (FIG. 23) and 22 kHz (FIG. 24) tone stimulations. The tonotopic responses appeared more ventromedial with the higher frequencies compared to the 11 kHz stimulus. Again, the Frings mice displayed significantly higher c-Fos immunoreactive cell counts within resulting tonotopic bands in the inferior colliculus compared to the AGS-resistant CF1 and C57BL/6J mice (FIG. 25). The average ratio of the density area was significantly greater for the Frings mice compared to the CF1 and C57BL/6J (see FIG. 26).

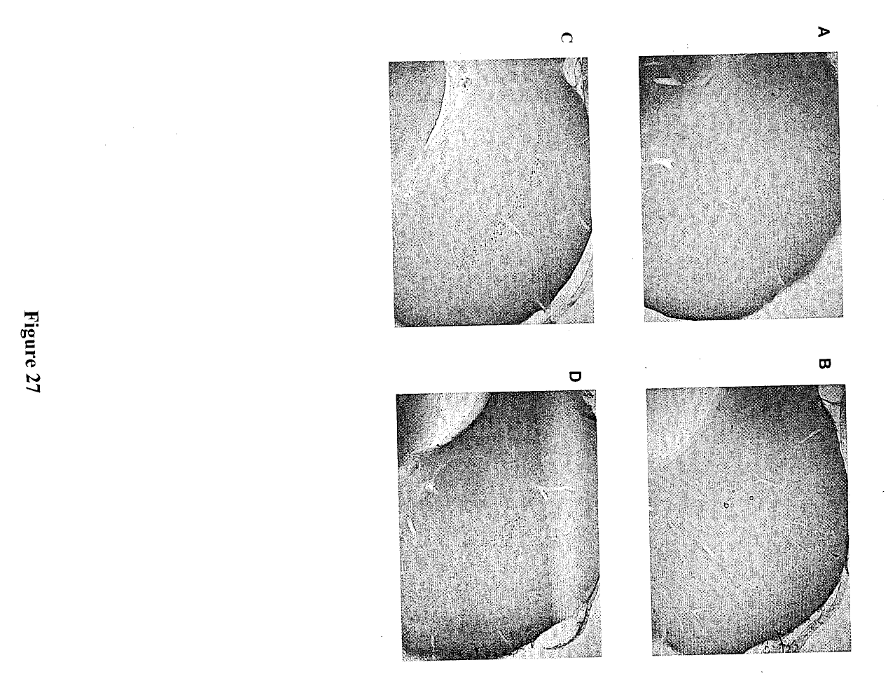

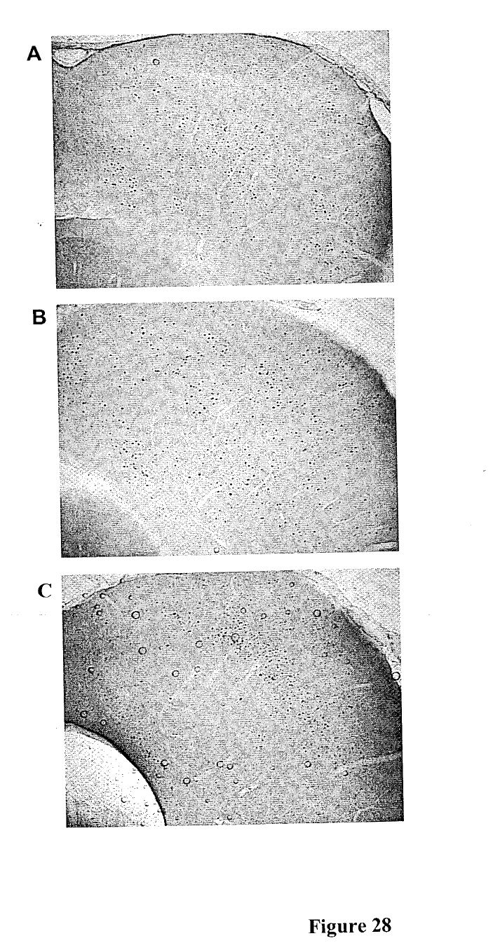

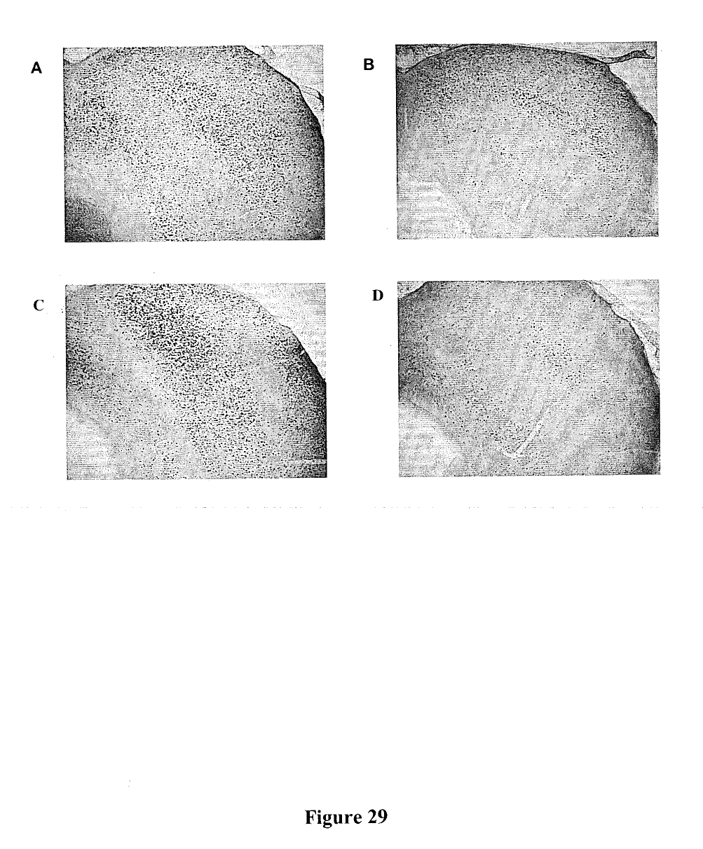

FIG. 27 displays the results from Frings, congenic, CF1 and C57BL/6J mice that were placed in the stimulation chamber, but with the speaker turned off. Almost no c-Fos immunoreactive cell staining was observed in the AGS-sensitive strains which also display elevated hearing impairment. However, the two AGS-resistant strains displayed focused, tonotopic responses which appeared to correspond to the position of the 16 kHz band. It was observed that the signal generator itself emits a very faint tone. The normal hearing, AGS-resistant, mice very likely responded to this faint background tone because maximum auditory sensitivity in mice is observed at 16 kHz (Shnerson and Pujol, 1983). FIGS. 28 and 29 show the response of Frings, SWR/Bm and C57BL/6J mice to an 11 kHz tone at different intensities. At 60 dB (FIG. 28) the Frings and SWR/Bm mice displayed only diffuse c-Fos immunoreactivity while the C57BL/6J mouse displayed a diffuse tonotopic response similar in appearance to the congenic and noise primed mice at the 80 dB intensity (FIG. 20). The 100 dB stimulation (FIG. 29) produced an AGS in the Frings mouse. The mouse was left in the stimulation chamber for the full 2 h to complete the tonotopic mapping study. The tonotopic density appears very high with heavy staining in the external nucleus and dorsal nucleus of the inferior colliculus and the periaqueductal grey indicating the initiation and propagation of an AGS. The SWR/Bm mouse showed heavy staining in the anterior medial section of the tonotopic band and in the external nucleus of the inferior colliculus, but no seizure was observed. Heavy staining was not observed in structures outside the inferior colliculus that are associated with AGS (FIG. 30) demonstrating that the pattern of neuronal activation in the inferior colliculus of the SWR/Bm mouse did not propagate outside the inferior colliculus (FIG. 30). The SWR/Bm mouse used in this study was heterozygous for the Frings mass1 deletion which may account for the neuroexcitability detected in the inferior colliculus from the prolonged 100 dB stimulation. The C57BL/6J mouse displays mostly diffuse staining with a faint tonotopic band. However, a congenic mouse exposed to the high intensity 11 kHz stimulus displays c-Fos immunoreactivity similar to Frings mice at 80 dB.

Overall, the AGS-sensitive mice, whether genetically or experimentally induced, displayed denser tonotopic responses with less c-Fos staining in adjacent areas of the inferior colliculi compared to the non-AGS susceptible mice. The more intense tonotopic c-Fos staining suggests a higher level of neuronal activation in the AGS-sensitive mice, even at the sub-AGS threshold stimulus intensity.

11. Behavioral Electroconvulsive Seizure Threshold (ECT) Testing

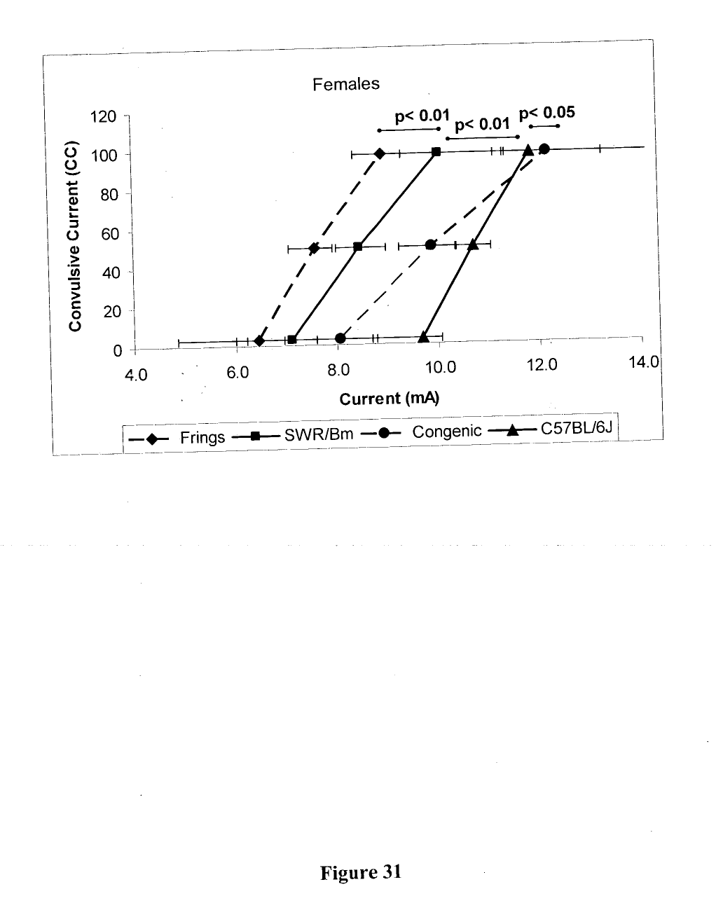

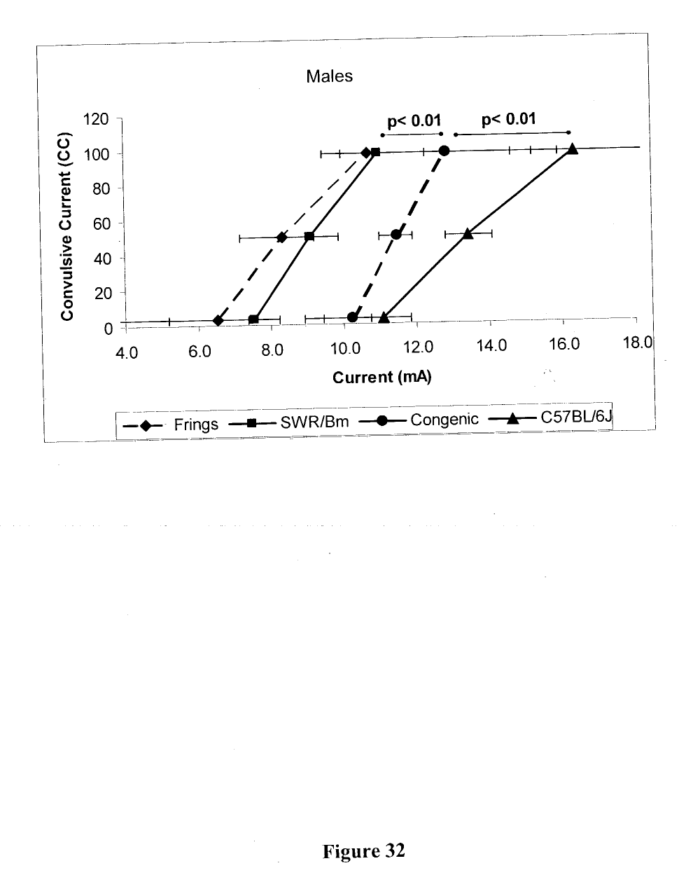

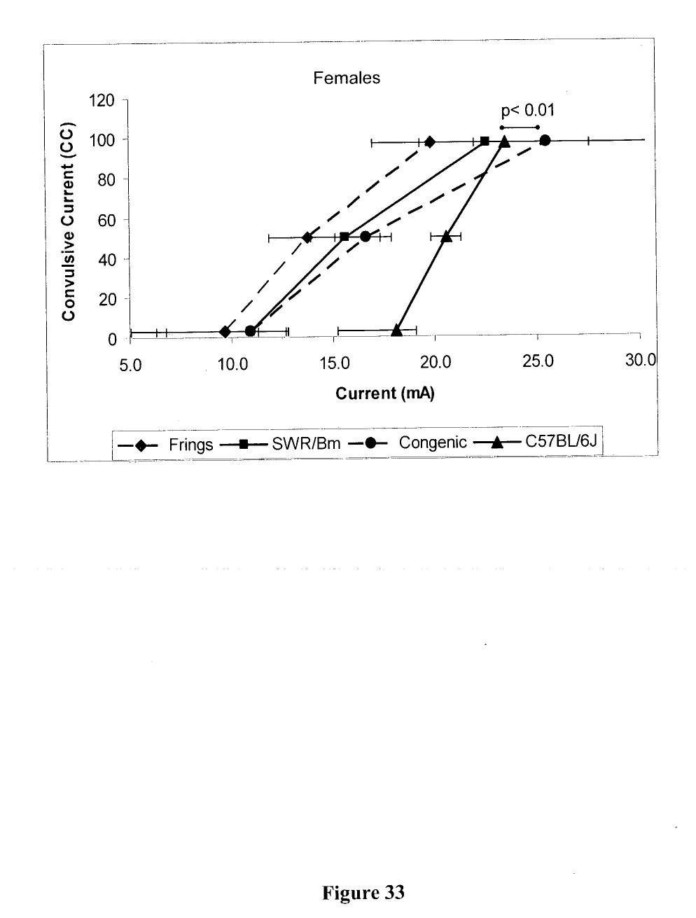

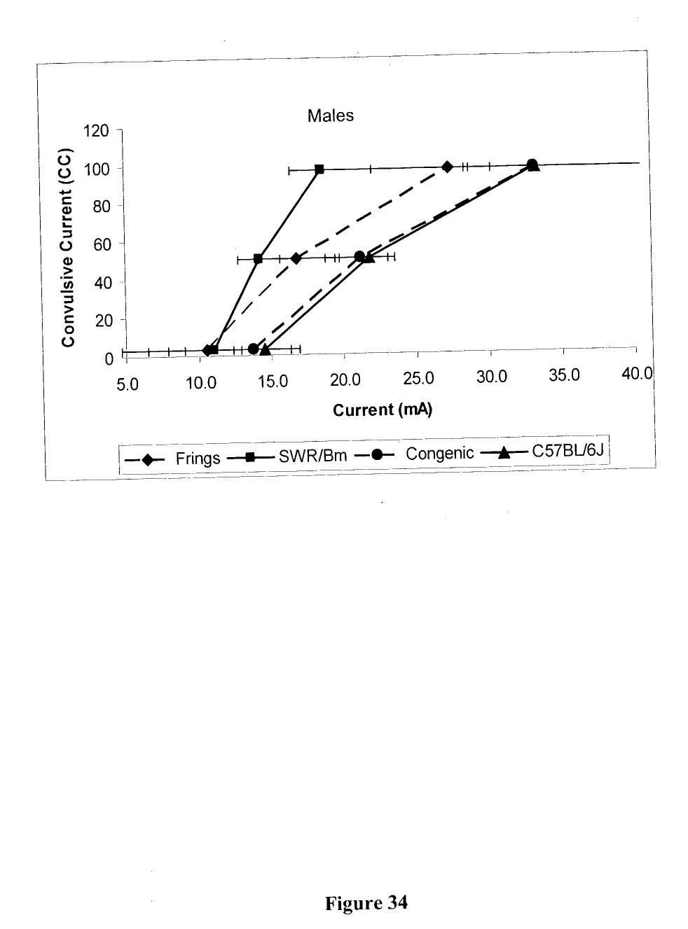

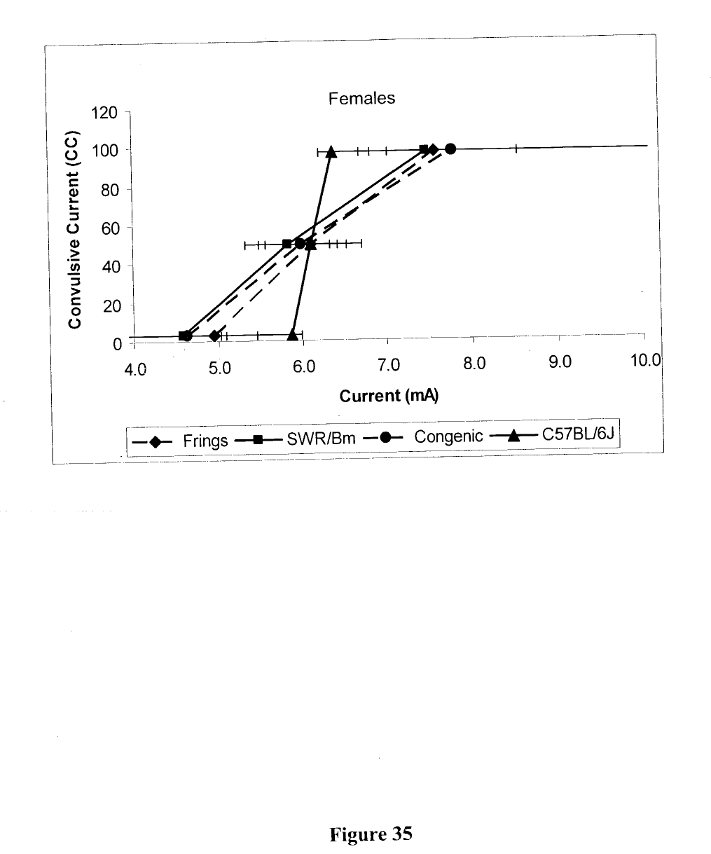

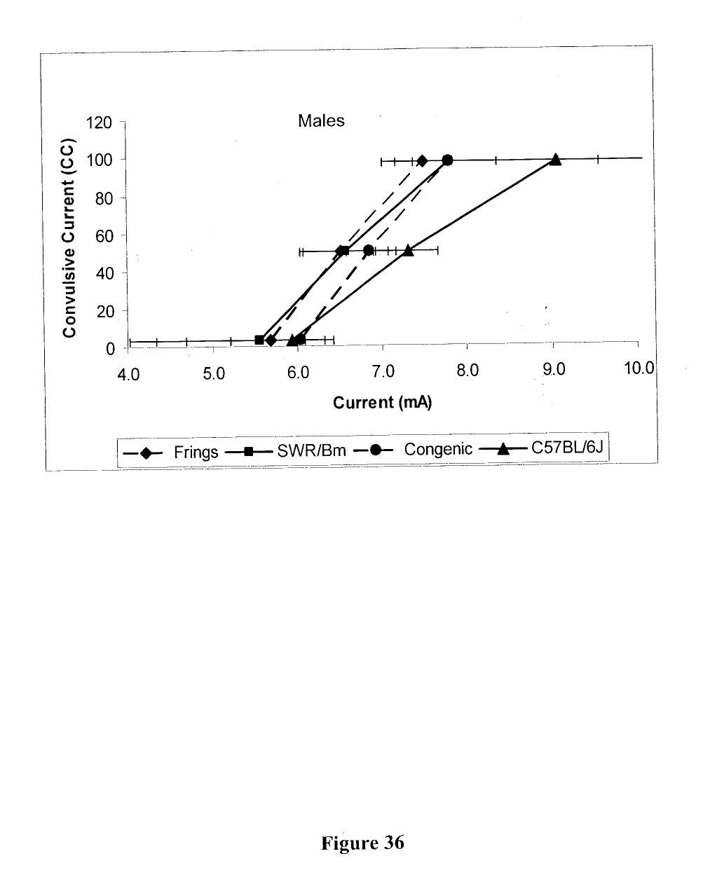

For each of the ECT tests, results obtained from Frings mice were compared to those from SWR/Bm mice, and results from C57BL/6J mice were compared to those from congenic mice (FIGS. 31, 32, 33, 34, 35, and 36). In general, the C57BL/6J and congenic mice exhibited higher ECTs than Frings and SWR/Bm mice, and male mice displayed higher thresholds than female mice within each strain. In the maximal ECT test, the congenic mice exhibited a significantly lower seizure threshold compared to the C57BL/6J mice (FIGS. 31 and 32), and the Frings female mice were lower compared to the SWR/Bm female mice (FIG. 31). For the psychomotor-partial ECT test, the congenic female mice displayed a significantly lower threshold compared to C57BL/6J female mice (FIG. 33). The minimal ECT test did not reveal a difference between any of the groups evaluated (FIGS. 35 and 36).

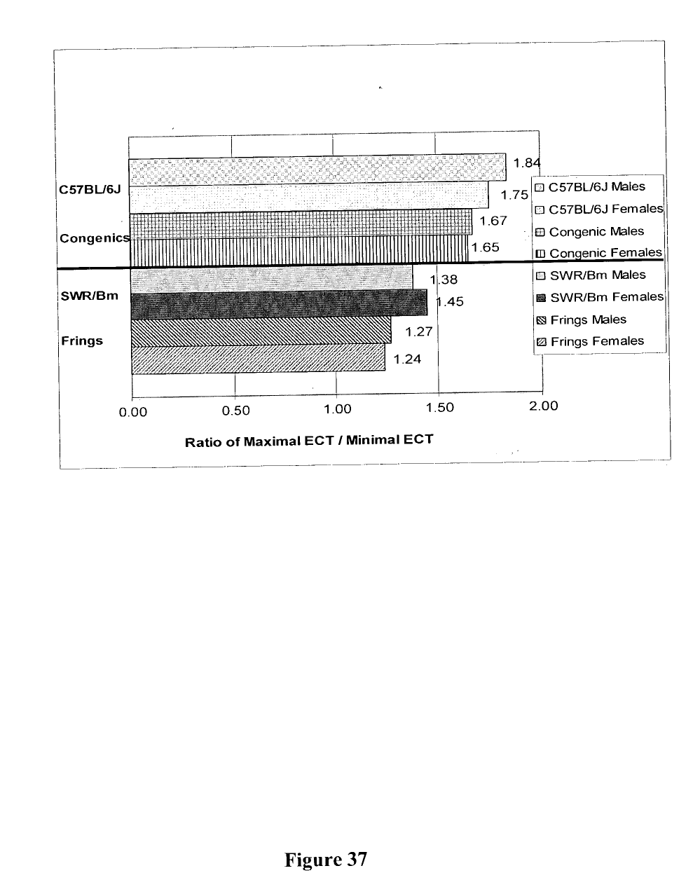

The ratio of maximal ECT/minimal ECT, at the CC50, was calculated for each of the strains as an indicator of propensity for seizure spread. In FIG. 37, a decreasing trend in the ratio of maximal ECT/minimal ECT was observed with the C57BL/6J and congenic mice displaying higher ratios than the SWR/Bm and Frings mice. Within each strain, except the SWR/Bm, the males showed a slightly higher resistance to seizure spread than the females. A decrease in the maximal ECT/minimal ECT ratio with Frings mice compared to SWR/Bm, and congenic mice compared to C57BL/6J mice, was observed suggesting a greater propensity for seizure spread in the mice homozygous for the mass1 gene deletion (FIG. 37).

All patents, publications, and commercial materials cited herein are hereby incorporated by reference.

### EXAMPLES

The following examples are given to illustrate various embodiments which have been made with the present invention. It is to be understood that the following examples are not comprehensive or exhaustive of the many types of embodiments which can be prepared in accordance with the present invention.

### Example 1

### Mouse breeding, seizure testing and DNA collection

Frings mice were crossed to the seizure-resistant strain C57BL/6J to produce F1 animals which, in turn, were intercrossed to generate 1200 F2 offspring. The Frings mice used in this study were bred in our colony and the C57BL/6J mice were supplied by the Jackson Laboratory (Bar Harbor, Me.). All mice were phenotyped at postnatal day 21 as seizure-susceptible or seizure-resistant as described previously. Skradski, S. L. et al., supra. Directly following seizure phenotyping, tail sections were cut for DNA preparation. Potential recombinant mice within the region were tested again to confirm the seizure phenotype, a second tail section was cut, and the mice were euthanized by CO₂ and bilateral thoracotomy. Spleens were harvested for DNA preparation by phenol/chloroform extraction and ethanol precipitation.

### Example 2

### Fine mapping

All known MIT microsatellite markers between cD13Mit200 and D13Mit126 were identified from the Chromosome 13 Committee map located at [http.://www.informatics.jax.org/ccr/searches/contents.cgi?&year=1999&chr.=13]. All F2 mice were initially tested with polymorphic markers D13Mit312, D13Mit97, and D13Mit69 to identify recombinant mice in the mass1 region, and the new recombinant mice were genotyped with additional markers, D13Mit9 and D13Mit190. Primer sequences and information for the markers was obtained from the Whitehead Institute Database site Genetic and Physical Maps of the Mouse Genome [http://www.genome.wi.mit.edulcgibin/mouse/index]. Primer synthesis and SSLP analysis was performed as previously described. Skradski, S. L. et al., supra.

### Example 3

### Yeast artificial chromosomes

YAC maps spanning the region were obtained from the Physical Maps of the Mouse Genome [http://www.genome.wi.mit.edu/cgi-bin/mouse/index]. YACs which appeared to contain SSLP markers known to be within the region were obtained from Research Genetics and YAC DNA was prepared by standard techniques. Haldi, M. L. et al. (1996), Mamm Genome 7: 767-9; Silverman, G. A. (1996), Methods in Molecular Biology, Vol. 54. D. Markie. Totowa, N.J., eds. Humana Press Inc. pp 65-68. All STSs shown to be associated with each YAC clone from the map were synthesized and tested to confirm that the clones were correct and aligned with overlapping YAC clones. Standard PCR conditions for physical mapping analyses were 10 mM Tris-HCl, 50 mM NaCl, 1.5 mM MgCl, 30 μM dNTPs, 0.5 μM of forward and reverse primers, and 50 ng of DNA in a 25 μL reaction volume. PCR thermocycles were 94° C. for 2 minutes, followed by 35-40 cycles of 94° C. for 10 seconds, 54° C. for 30 seconds, and 72° C. for 30 seconds with a 5 minute final extension at 72° C.

### Example 4

### Bacterial artificial chromosomes

BACs were identified and isolated from the PCR-based mouse BAC library available from Research Genetics using all known STSs and SSLPs found in the region on linkage and YAC maps. BAC DNA was prepared using purification columns by the recommended procedure (Magnum columns, Genome Systems, Inc). BAC end sequence was obtained using T7 and SP6 primers. Individual BAC insert sizes were determined by complete digestion of the BAC DNA with NotI and separating the fragments on a 1.0% agarose gel in 0.5× TBE circulating buffer. The field inversion gel electrophoresis (FIGE) program was 180 volts forward, 120 volts reverse, 0.1 seconds initial switching time linearly ramped to 3.5 seconds switching time for 16 hours.

### Example 5

### Simple sequence length polymorphism (SSLP) identification

BAC DNA was partially digested with Sau3AI into fragments ranging from 1 to 3 Kb and subcloned into the Bam I site of pUC 18 with the Ready-To-Go cloning kit (Amersham Pharmacia Biotech). New repeats were identified by plating the subclone library, lifting duplicate Hybond-N membranes (Amersham Pharmacia Biotech), and hybridizing with (CA)₂₀ and (AT)₂₀ oligonucleotides end-labeled with γ³²P-ATP. Hybridized membranes were exposed to autoradiographic film. Clones producing a positive signal were sequenced and primer pairs were designed to amplify new repeat sequences. New SSLP markers were tested with control and recombinant mice to finely map. the interval.

### Example 6

### Cosmid subcloning

BAC 290J21 was partially digested with Sau3AI into 30-40 Kb fragments which were subcloned into cosmids as per the instructions for the SuperCos 1 cosmid vector kit (Stratagene) and packaged with Gigapack III Gold Packaging Extract (Stratagene) using XL1-Blue mrf competent cells. Cosmids were then aligned by amplification with all STSs across the region. Cosmid sequencing was performed by standard techniques using 1200 ng of cosmid DNA and 3.2 pmole of gene-specific mass1 oligos ranging from 18 to 24 nucleotides in length.

### Example 7

### Identifying and cloning the mass1 gene

The mass1 cDNA was identified by reverse transcription-PCR.(RT-PCR) using primers developed from sequence of exons predicted by Genefinder [http://dot.imgen.bcm.tmc.edu:9331/gene-finder/gf.html]. Total RNA was prepared from whole mouse brain of C57BL/6J, Frings and F1 mice with Trizol reagent as per instructions (Molecular Research Center, Inc.). The standard reverse transcription reaction conditions were 1.0 μg RNA, 15 ng random hexamers, 1× First Strand Buffer, 10 mM DTT, I mM dNTPs, 40 U RNAse Inhibitor, and 200 U Superscript II reverse transcriptase (Gibco BRL). First strand cDNAs were amplified using pfx DNA polymerase (Gibco BRL) and multiple reactions were sequenced for each. Since the entire gene was not contained within the genomic sequence that was generated, 5′- and 3′-RACE was used to identify the remaining cDNA sequences.

### Example 8

### Reverse transcription-PCR

The RT reactions to determine tissue specificity of mass1 expression were performed as described in the previous section on samples from CF1 (Charles Rivers, Wilmington, Mass.), C57BL/6J (The Jackson Laboratory, Bar Harbor, Me.), or Frings mouse tissues and cells. The tissue panel samples were isolated from a single C57BL/6J mouse. The neuronal cDNA was produced from the pooled cellular extracts of 4-6 CF1 mouse cultured cortical neurons, and the astrocyte cDNA from CF1 astrocyte culture RNA extracted with Trizol reagent (Molecular Research Center, Inc). PCR conditions to amplify the cDNAs were 10 mM Tris-HCl, 50 mM KCl, 1.5 mM MgCl, 30 μM dNTPs, 0.5 μM of forward and reverse primers, and 1 μL of the cDNA in a 25 μL reaction volume. PCR thermocycles were 94° C. for 2 minutes, followed by 25 (β-actin primers) or 40 (mass1 primers) cycles of 94° C. for 10 seconds, 54° C. for 30 seconds, and 72° C. for 30 seconds with a 5 minute final extension at 72° C. The mass1 primers spanned from exon 22 to exon 23, the forward was 5′ CAG AGG ATG GAT ACA GTA C 3′ (SEQ ID NO: 29) and the reverse was 5′ GTA ATC TCC TCC TTG AGT TG 3′ (SEQ ID NO: 30) and the expected product size was 487 base pairs. Theβ-actin primers also spanned an intron and were forward 5′ GCA GTG TGT TGG CAT AGA G 3′ (SEQ ID NO: 31) and reverse 5′ AGA TCC TGA CCG AGC GTG 3′ (SEQ ID NO: 32) and the expected product size was 327 base pairs. PCR products for. each tissue were mixed and separated by gel electrophoresis on 2% agarose gels in 1× TAE buffer at 120V,. and the bands visualized by staining with ethidium bromide using an ultraviolet (UV) light source.

### Example 9

### Polymorphism and mutation identification

For SSCP, the mouse DNA samples A/J, AKR/J, BALB/cJ, C57BL/6J, C3H/HeJ, CAST/EiJ, LP/J, NON/LtJ, NOD/LtJ, SPRET/EiJ, and DBA2/J were supplied by the Jackson Laboratory (Bar Harbor, Me.). The CF1 mice were supplied by Charles Rivers (Wilmington, Mass.), and the seizure-susceptible EL, EP, and SAS mice were supplied by Dr. T. Seyfried (Boston College, Boston, Mass.). PCR reactions were identical to those conditions listed above except 0.3 μL of α³²P-dCTP was included in a 10 μL total reaction volume. A 30 μL aliquot of dilution buffer (0.1% SDS/10 mM EDTA in ddH₂O) was added to the PCR reactions. A 10 μL aliquot of the dilute PCR reaction was mixed with 10 μL of loading dye (bromophenol blue/xylene cyanol) and 2 μL samples were separated by non-denaturing electrophoresis on an 9% bis-acrylamide, 10% glycerol, non-denaturing gel at 20W for 14 hours at room temperature with a fan. The PCR forward primer sequence was 5′ TTT ATT GTA GAG GAA CCT GAG 3′ (SEQ ID NO: 33) and the reverse primer sequence was 5′ GCC AGT AGC AAA CTG TCC 3′ (SEQ ID NO: 34) and the expected product size was 126 base pairs. Exon 27 PCR products were sequenced to determine that the aberrant band was due to a single G deletion in the Frings mouse mass1 gene as shown for C57BL/6 and Frings mouse DNA.

### Example 10

### MASS1 amino acid sequence analysis

The amino acid sequence of MASS1 was deduced from the nucleotide sequence of the cloned mass1 cDNA by DNA Star. The amino acid sequence was compared to known proteins by BLAST sequence similarity searching [http://www.ncbi.nlm.nih.gov/blast/blast.cgi]. Identification of functional domains utilized PSORT II Prediction [http://psort.nibb.acjp/form2.html], Sequence Motif Search [http://www.motif.genome.adjp/], Global and Domain Similarity Search [http://wwwnbrf.georgetown.edu/pirwww/search/dmsim.html], and Pattern Match. [http://www-nbrf.georgetown.edu/pirwww/search/patmatch.html].

### Example 11

### Identification and Mapping of a BAC containing the hMass1 gene

Human mass1 was detected by a relaxed RT-PCR. Several primer sets corresponding to different exons of mouse mass1 were used to amplify human fetal brain cDNA. PCR conditions were the same as in mouse amplifications with an exception of the annealing temperature of 47° C. These primers were used to identify a human genomic clone containing a part of the hmass1 gene (CITB human BAC library).

Human lymphoblast cultures were treated with 0.025 mg/ml cholcimid at 37° C. for 1.5 hr. Colcimid treated cultures were pelleted at 500×g at room temperature for 8 min. Pellets were then re-suspended with 0.075M KCl, 3 ml per pellet 15 minutes at room temperature. Cells were then fixed in 3:1 MeOH:acetic acid and stored at 4° C. Human BACs were labeled with spectrum orange using a nick translation kit per the manufacturer's protocol (Vysis, Downers Grove, Ill.). Slides were prepared by dropping fixed cells onto glass slides and washing with excess fixative. The slides were then washed in acetic acid for 35 min at room temperature and dehydrated in 70%, 85%, and finally 100% EtOH (2 min each). Chromosomes were denatured in 70% formamide in 2×SSC at 74° C. for 5 minutes and slides were dehydrated again as above except in ice cold EtOH. Two μg of labeled probe was blocked with 2 μg of human Cot-1 DNA in Hybrisol VI (ONCOR, Gaithersburg, Md.). The probe mixture was denatured at 74° C. for 5 minutes and then pre-annealed at 37° C. for 15 min. Twelve μL of pre-annealed probe was applied per slide, a cover slip was added and edges were sealed with rubber cement. Slides were hybridized in a darkened, humidified chamber for 16 hr at 37° C. Hybridized slides were then washed in 0.4× SSC containing 0.1% Tween-20 at 74° C. for 2 min, followed by 1 min at room temperature in 2× SSC. Slides were allowed to dry in the dark at room temperature and were stained with DAPI (Vector labs, Burlingame, Calif.) for chromosome visualization.

### Example 12

### Establishment of Breeding Colony of Frings mass1-Homozygous Mice

BUB/bnJ, SWR/Bm, C57BL/6J and DBA/2J mice were purchased from The Jackson Laboratory and small colonies were established and maintained by the- University of Utah Animal Resource Center until the day of the experiments. CF1 mice were purchased from Charles River laboratories. The Frings mice were obtained from an in-house colony at the University of Utah that has been maintained for over 30 years. For the congenic strain, Frings mice (donor allele) were crossed with the seizure-resistant C57BL/6J mice as the recipient strain. The (Frings X C57BL/6J) F1 progeny was back crossed to the C57BL/6J parental strain. Subsequent (N2-N4) generations were genotyped (Skradski et al., 2001) and those with the Frings mass1 allele were backcrossed to the C57BL/6J parental strain. The N5 generation was intercrossed and progeny that were homozygous for the Frings mass1 allele were used to establish breeding pairs to produce a small colony for the subsequent ABR, AGS, and ECT testing. All animals were allowed free access to food and water and were housed in a temperature- and light-controlled environment (12-hr on/12-hr off).

### Example 13

### Measurement of Auditory Brainstem Response Thresholds

Mice from strains genetically AGS-sensitive (Frings, DBA/2J, BUB/bnJ, congenic) and AGS-resistant (C57BL/6J, SWR/Bm) and experimentally-induced for AGS-susceptibility (noise primed and kanamycin treated C57BL/6J) were evaluated at various age-points from pre-weanling to advanced age for ABR thresholds as a measure of auditory function. ABR thresholds were measured using instrumentation and software from Intelligent Hearing Systems (SmartEP version 2.39, Opti-Amp 3000D Pre-amplifier - IHS, Miami, Fla.). Mice were anesthetized-with Avertin solution administered i.p. at a dose of 0.02 ml per gram of body weight plus an additional 0.1 ml. Acoustic stimulation was presented though a pair of high-frequency transducers (IHS) for clicks and tone burst acoustic stimulations at 10 kHz, 16 kHz and 22 kHz. Acoustic click stimulation was presented binaurally at 50 μs alternating polarity at a rate of 29.1/s for a total of 1024 stimuli. Tone bursts were presented for 3000 μs using the exact Blackman waveform. ABR thresholds were measured using sub-dermal electrodes placed ventrolateral to each ear and a ground electrode placed at the forehead. One channel, using the electrode under the right ear, was recorded with the bandpass filtered below 100 Hz and artifact rejection set at 31 μV. Acoustic intensity was usually started at 60 dB or 80 dB and increased or decreased at 10 dB steps until near the ABR threshold. The ABR threshold was then bracketed using 5 dB steps. ABR threshold was determined by comparing ABR patterns on the screen and the lowest level at which an ABR pattern could be recognized was recorded as the threshold. The threshold was usually bracketed by two subthreshold and several suprathreshold intensities.

### Example 14

### Assessment of Audiogenic Seizure Susceptibility

Mice were tested for AGS-sensitivity using either an 11 kHz tone or an electric bell stimulus. For the 11 kHz stimulus, mice were placed in a cylindrical clear plastic chamber and an 11 kHz tone at 110 dB was presented for 60 s or until tonic extension was elicited. The acoustic presentation was controlled by the MurSon software version 2.0 by Ztech. AGS severity was scored with the following scale; 0 for no response, 1 for wild running only (<10 s), 2 for wild running only (>10 seconds or 2 bouts of wild running), 3 for wild running with loss of righting reflex, 4 for clonus, and 5 for tonic hindlimb extension. For the electric bell stimulation, an electric doorbell attached to a wire mesh frame was mounted over a clear rectangular plastic mouse cage. Sound intensity was presented at 110 dB SPL and measured using a sound pressure level meter (Bruel and Kjaer Model Type 2231).

### Example 15

### Experimental Induction of Audiogenic Seizure Susceptibility

Kanamycin: To produce ototoxicity, C57/BL/6J mice were dosed daily between PND 6 and PND 21 with 400 mg/kg kanamycin sulfate (Sigma) diluted in saline and administered i.p. Littermate control mice were injected with an equal volume of saline. Kanamycin-treated mice were evaluated for ABR thresholds at PND 19 and PND 24 and tested for AGS sensitivity on PND 29, PND 41 and PND 49.

Noise Priming: Noise priming of young, pre-weanling mice was conducted using the electric bell and chamber described above. C57BL/6J mice between PND 16 and PND 20 were exposed to the electric bell stimulation of 110 dB for 30 or 120 s. None of the C56BL/6J mice displayed AGS-sensitivity during the noise priming. The mice were then tested for AGS-sensitivity between PND 28 and PND 41 using the electric bell at 110 dB for 60 s.

### Example 16

### Tonotopic Mapping

Acoustic Stimulation: Mice (5 to 6 weeks-old) were exposed to a continuous sub-seizure threshold tone stimulation at 11 kHz (80 dB), 16 kHz (78 dB) or 22 kHz (80 dB) for 90-120 minutes. Any AGS-sensitive animals that displayed a seizure were removed from the study except as indicated in the results. During the acoustic exposure, mice were placed in a cylindrical clear plastic chamber with a speaker mounted on top. The bottom of the chamber was a wire screen to support the mice and the chamber was elevated one meter above acoustic dampening foam to scatter emitted sound waves and reduce resonance within the chamber. The tone stimulation was produced using an HP Model 200CD wave generator. Sound intensity was measured using a Bruel and Kjaer Model Type 2231 sound pressure level meter.

c-Fos Immunohistochemistry: The immediate early gene product, c-Fos, was used as a marker of neuronal activation to reveal tonotopic response domains in the inferior colliculus. Immediately following acoustic stimulation the brains- were processed for c-Fos immunohistochemistry. The mice were perfused intracardially under deep ketamine/xylazine anesthesia with phosphate-buffered saline (PBS) followed by 4% paraformaldehyde in PBS. Following perfusion the brains were post-fixed and sliced into 50 μm coronal sections using a vibratome.

c-Fos immunohistochemistry was performed on free-floating brain slices. The sections were pretreated for 0.5 h with 0.5% solution of H₂O₂ and blocked with 4% inactivated normal goat serum for 0.5 h in PBS/0.3% Triton X-100 prior to rinse with normal PBS. The primary antibody (Ab2, Oncogene Research Products, Cambridge, Mass.) was diluted 1:1000 in PBS with 1% bovine serum albumin (BSA)/0.3% Triton X-100 and incubated overnight (18-24 hours) at 4° C. The biotinylated goat anti-rabbit IgG (Oncogene Research Products) was diluted 1:400 in PBS with 1% bovine serum albumin (BSA)/0.3% Triton X-100. The sections were then incubated in avidin-biotin-horseradish peroxidase (Vectastain Elite ABC kit, Vector Laboratories Inc.) for lh. Labeling was revealed by exposure to 3,3′-diaminobenzidine DAB substrate (Peroxidase Substrate kit, Vector Laboratories Inc.) for 10-15 minutes. c-Fos immunoreactivity was visualized as a brown staining in the cell nuclei.

### Example 17

### Electroconvulsive Seizure Threshold Testing

Regional neuronal excitability was assessed by electroconvulsive-induced minimal clonic, maximal tonic and psychomotor-partial seizures. The electrical threshold for each seizure-type was determined by passing electrical current into the brain using transcorneal stimulating electrodes. A drop of saline with 0.5% tetracaine was applied to each eye prior to stimulation. For the minimal and maximal ECT, the electrical current was controlled with an apparatus described by Woodbury and Davenport (1952) and utilizing a sinusoidal wave pulse at 60 Hz, 16 ms pulse width and 0.2 s duration. Minimal clonic seizures were characterized by rhythmic jaw and forelimb clonus, and could include ventral flexion of the neck, rearing and falling. A maximal tonic hindlimb seizure was recorded if the hindlimbs passed 90 degrees to the plane of the body. For the psychomotor-partial seizures, current was controlled using a Grass stimulator Model S4B and was presented at 6 Hz, 0.2 ms pulse width for a 3.0 s duration. Psychomotor partial seizures were characterized by stun and rapid rhythmic movement of the vibrissae and could include jaw and forelimb clonus, neck flexion, and rearing and falling.

The ECT for each test was measured separately for both genders of Frings, SWR/Bm, C57BL/6J and congenic mice that were between 6 and 10 weeks postnatal. Population ECT was determined via the staircase procedure as described by Finney (1971). Briefly, the stimulation intensity for an animal was determined by the response (defined by the presence of a seizure) or lack of response of the previous animal. The convulsant current (CC) required to produce a seizure in 3% (CC3), 50% (CC50) and 97% (CC97) of the population was calculated by Probit analysis (Finney, 1971). Statistical significance between groups was determined using regression tables in the Probit analysis using the MINITAB statistical software.

Analysis

Images of the inferior colliculus processed for c-Fos immunohistochemistry were captured at 40× magnification and analyzed using NIH Image software (version 1.57; NIH, http://www.rsb.info.nih.gov/nih-image1). c-Fos immunoreactive cells were manually counted, with the image label covered, using a template overlaid on each captured image to define the region of interest. The region of interest was drawn as a band within the central nucleus of the inferior colliculus corresponding to the tonotopic domain for each of the tested frequencies. The boundaries for the central nucleus were determined using the mouse brain atlas (Franklin and Paxinos, 1997). The templates were used to quantify c-Fos positive cell counts and to compare the area of c-Fos immunoreactive staining (in pixels) within the tonotopic frequency domain, to the same size areas immediately above and below the response domains. For pixel area analysis, the density slice mode in the NIH Image software was utilized. The upper threshold was always set at the maximum (255), and the minimal threshold was adjusted within a range of 95 to 150 to minimize background. The pixel area (number of pixels with density falling between the upper and lower thresholds) was measured and results reported as the ratio of the pixel area within the tonotopic band divided by the average pixel area of the two immediately adjacent areas. Statistical analysis was preformed using GraphPad Prism version 3.02 (GraphPad Software Inc.). Results for the c-Fos positive cell counts were compared using one-way ANOVA and Tukey's post-hoc analysis. Pixel area ratios were compared for statistical significance using the Kruskal-Wallis test and Dunn's post-hoc analysis.

### SUMMARY

In summary, a novel gene which is associated with the Frings phenotype in mice has been isolated and characterized. The gene is known as the Monogenic Audiogenic Seizure-susceptible gene or mass1. The product of the mass1 gene is designated MASS1. Nucleic acid molecules that encode for MASS1 have been identified and purified. The sequence of murine mass1 can be found at SEQ ID NO: 1, and the sequence of human mass1 can be found at SEQ ID NO: 3. Mammalian genes encoding a MASS1 protein are also provided. The invention also provides recombinant vectors comprising nucleic acid molecules that code for a MASS1 protein. These vectors can be plasmids. In certain embodiments, the vectors are prokaryotic or eukaryotic expression vectors. The nucleic acid coding for MASS1 can be linked to a heterologous promoter. The invention also relates to transgenic animals in which one or both alleles of the endogenous mass1 gene is mutated.

In addition to the above, the invention relates to a hearing impairment associated with the Frings mass1 mutation. More specifically, the invention characterizes a moderate and non-progressive hearing impairment measurable with the Auditory Brainstem Response (ABR) technique. This hearing impairment leads to the development of audiogenic seizures.

Measuring auditory brainstem responses demonstrated that the Frings mass1 gene produces a moderate and relatively stable, very early onset, hearing impairment in Frings mice. Without being limited to any one theory, these characteristics of the Frings hearing impairment, resulting from the mass1 gene mutation, appear to be critical to the robust AGS phenotype. The congenic and BUB/bnJ-mice, which also possess the Frings mass1 alleles, display very early-life hearing impairment similar to the Frings mice. However, the hearing impairment with these strains rapidly progresses, causing them to lose AGS-susceptibility within two months postnatal. The relatively stable hearing impairment in the Frings mice is unusual. In an ABR screening of 80 inbred mouse strains, 34 strains were found to display early or late-onset hearing loss and all were progressive (Zheng et al., 1999). Therefore, the Frings mouse, which displays a relatively stable hearing impairment phenotype, is not typical.

Results from the auditory studies demonstrate that the Frings mouse, with the mass1 gene deletion, may provide a new model for hereditary hearing loss. Genetic hearing impairment is a significant disease that affects about 1 in every 2000 children (Morton, 1991). Typically, single gene mutations inherited in a predictable Mendelian fashion are involved in hereditary hearing impairment in children (Battey, 2001). The mouse is recognized as an excellent animal model for the study of heredity human deafness and the National Institute on Deafness and Other Communication Disorders gives high priority to research to understand the genes involved in hereditary hearing impairment (Battey, 2001). Therefore, the Frings mouse with the mass1 gene mutation, as an identified single gene defect with predictable Mendelian transmission, may represent a valuable new genetic model for studying heredity hearing impairment.

A rapid maturation of central auditory structures occurs following hearing onset, which in mice is about PND 13 (Chen and Willott, 1983). Hearing impairment during this critical period has been shown to alter maturation of neuronal circuits in the inferior colliculus (Chen and Willott, 1983; Li et al., 1994; Pierson and Snyder-Keller, 1994; Kwon and Pierson, 1997). Hearing impairment associated with the Frings mass1 gene deletion was detected as early as PND 15, which was the earliest age tested (Table 3.1), demonstrating that it causes a loss of auditory input during the critical period following hearing onset.

Sound-induced neuronal responses in the inferior colliculus demonstrated that a significant hyper-responsiveness within tonotopic bands develops in Frings mice compared to AGS-resistant mice (FIGS. 3.7-3.14). Greater neuronal activation was observed in tonotopic domains of the DBA/2J, and experimentally-induced C57BL/6J mice compared to AGS-resistant mice. However, the difference observed with the later strains was not always statistically significant. These studies demonstrate that the robust AGS phenotype in Frings mice is likely associated with a significant hyper-responsiveness in the inferior colliculus.

The brain structures involved in the Frings AGS were determined by detection of c-Fos expression. c-Fos expression is a useful technique for determining which brain structures display neuronal activation in response to seizures, but may only give a rough indication of the level of activity. AGS-associated neuronal activity in Frings mice appeared to be mostly limited to a brainstem seizure network.

Behavioral ECT testing was used to measure regional neuroexcitability (brainstem, forebrain and limbic structures) associated with the Frings MASS1 gene deletion. The ECT tests in the Frings and congenic mice demonstrated that the mass1 gene deletion is associated with a lowered threshold for maximal ECT. Furthermore, a decrease in the ratio of the maximal ECT/minimal ECT was observed. These results demonstrate that the Frings mouse displays a greater propensity for seizure spread. This may suggest that the Frings mass1 deletion exerts a direct effect on intrinsic neuroexcitability in the brainstem. Therefore, the robust AGS-susceptibility in Frings mice that develops from the early onset hearing impairment appears to occur on a genetically predisposed seizure background. AGS-susceptibility in several mouse strains corresponds to the threshold for maximal ECT. Mouse strains with the lowest maximal ECT and ratio of maximal ECT/minimal ECT displayed the highest penetrance for AGS. This finding indicates that the ability to develop AGS-susceptibility in mice may provide a good model for investigating the influence of genetic intrinsic neuronal excitability on the development of generalized epilepsy.

Whether the mass1 transcripts are alternative transcripts of VLGR1, or they encode a protein with a separate function, the embryonic expression pattern may suggest a role in the developing CNS. The proposed involvement of VLGR1 in cell migration (McMillan et al., 2002) is particularly intriguing. Altered neuronal cell migration during development could produce lasting effects on intrinsic neuroexcitability, consistent with the changes observed in the brainstem of adult mice with the mass1 deletion. Furthermore, the normal function of the inner ear depends on the migration of a number of cell-types from the neural crest to the developing inner ear, including melanocytes (Steel et al., 1983). For this reason, mutations in spotting genes that result in areas devoid of neural crest-derived melanocytes, suggesting a possible defect with migration, are frequently associated with heredity deafness (Steel et al., 1983). Therefore, alterations in cell migration may provide an explanation for the pathophysiology associated with the Frings mass1 deletion, both for the hearing impairment and the brainstem neuroexcitability. Previously characterized mouse genetic seizure models appear to be caused by mutations to ion channels (Noebels, 2000; Ptacek and Fu, 2001). The predicted protein sequence from the MASS1 transcript shares no homology with any identified ion channels. Without being limited to any one theory, this may suggest a novel function (Skradski et al., 2001).

The invention may be embodied in other specific forms without departing from its essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

We claim:

1. A transgenic mammal wherein one or both alleles of the endogenous mass1 gene is mutated, wherein the mutation results in an early-onset hearing impairment phenotype.

2. The transgenic mammal of claim 1, wherein the transgenic mammal is a mouse.

3. A cell derived from the transgenic mammal of claim 1.

4. A method for inducing early-onset hearing impairment in a mammal comprising mutating one or both alleles of the endogenous mass1 gene in the mammal.

5. The method of claim 4, wherein mutating one or both alleles of the endogenous mass1 gene in a mammal comprises inserting a selectable marker gene sequence or other heterologous sequence into the genome by homologous recombination.

6. The method of claim 4, wherein mutating one or both alleles of the endogenous mass1 gene in a mammal results in the production of a truncated MASS1 protein.

7. The method of claim 4, wherein mutating one or both alleles of the endogenous mass1 gene in a mammal comprises deleting guanine at position ***** of the endogenous mass1 gene, thus producing a truncated MASS1 protein.

8. The method of claim 4, wherein the mammal is a mouse.

9. A method of evaluating the potential therapeutic value or potential medical significance of a proposed anticonvulsant agent comprising the steps of providing the proposed anticonvulsant agent to a transgenic mammal wherein one or both alleles of the endogenous mass1 gene are mutated, and examining the therapeutic value or medical significance of the proposed anticonvulsant agent in the transgenic mammal.

10. The method of claim 9, wherein the transgenic mammal is a mouse.

11. The method of claim 10, wherein the transgenic mammal is a Frings mouse.

12. The method of claim 9, wherein the step of examining the therapeutic value or medical significance of the proposed anticonvulsant agent in the transgenic mammal comprises exposing the transgenic mammal to intense acoustic stimulation and observing whether the mammal experiences a seizure.

13. The method of claim 12, wherein the transgenic mammal is a mouse.

14. The method of claim 13, wherein the transgenic mammal is a Frings mouse.

15. A method of evaluating the potential therapeutic value or potential medical significance of a proposed agent for use in hearing impairment therapies comprising the steps of providing the proposed agent to a transgenic mammal wherein one or both alleles of the endogenous mass1 gene are mutated, and examining the therapeutic value or medical significance of the proposed anticonvulsant agent in the transgenic mammal.

16. The method of claim 15, wherein the transgenic mammal is a mouse.

17. The method of claim 16, wherein the transgenic mammal is a Frings mouse.

18. The method of claim 15, wherein the step of examining the therapeutic value or medical significance of the proposed agent for use in hearing impairment therapies in the transgenic mammal comprises observing the auditory brainstem response of the mammal to an auditory stimulus.

19. The method of claim 18, wherein the transgenic mammal is a mouse.

20. The method of claim 19, wherein the transgenic mammal is a Frings mouse.
